(19) United States
(12) Patent Application Publication
(10) Pub. No.: US 2023/0414793 A1
(43) Pub. Date: Dec. 28, 2023

(54) NANOPARTICLE

(71) Applicant: Sumitomo Chemical Co., Ltd., Tokyo (JP)

(72) Inventors: Tania Zuberi, Godmanchester (GB); Sheena Zuberi, Godmanchester (GB); Kiran Kamtekar, Godmanchester (GB)

(73) Assignee: Sumitomo Chemical Co., Ltd., Tokyo (JP)

(21) Appl. No.: 18/035,273

(22) PCT Filed: Nov. 4, 2021
(86) PCT No.: PCT/EP2021/080696

§ 371 Date: May 3, 2023

(30) Foreign Application Priority Data
Nov. 4, 2020 (GB) 2017440.5
Sep. 9, 2021 (GB) 2112888.9

(51) Int. Cl. A61K 49/22; A61K 49/00; B82Y 20/00; B82Y 15/00

(52) U.S. Cl. CPC A61K 49/225; B82Y 15/00; B82Y 20/00; A61K 49/0004

(57) ABSTRACT

Nanoparticles for use in diagnosis by photoacoustic imaging or therapy by photothermal therapy are disclosed. The nanoparticles may have a core containing a light-absorbing material and silica. The nanoparticles may contain a light-absorbing material comprising an electron accepting unit and an electron donating unit wherein the electron-donating unit is a unit of formula (IIIa-1) wherein: Y in each occurrence is independently O or S; Z in each occurrence is O, S, NR⁵⁵, or C(R⁵⁴)₂; R⁵¹ in each occurrence is H or a substituent; R⁵⁴ in each occurrence is independently a substituent; and R⁵⁵ is H or a substituent.

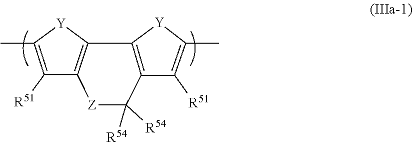

Embodiments of the present disclosure relate to particles for use in photoacoustic imaging (PAI) or photothermal therapy (PTT) and to use of said particles in PAI or PTT.

In PAI, light pulses are delivered into a targeted area of biological tissue where it is absorbed. Light energy converted to heat within the tissue results in thermoelastic expansion and acoustic emission which may be detected by suitable apparatus, e.g., an ultrasonic transducer, and converted to an image of the targeted area.

In PTT, heat generated following absorption of the light energy may kill targeted cancer cells.

Xiaoju Men and Zhen Yuan, “Multifunctional conjugated polymer nanoparticles for photoacoustic-based multimodal imaging and cancer photothermal therapy”, Journal of Innovative Optical Health Sciences Vol. 12, No. 03, 1930001 (2019) discloses PAI using conjugated polymer nanoparticles formed by a self-assembly, mini-emulsion or non-reprecipitation method.

Yang et al, “A 1064 nm excitable semiconducting polymer nanoparticle for photoacoustic imaging of gliomas”, Nanoscale, 2019, 11, 7754-7760 discloses a polymer nanoparticle as a potential contrast agent for photoacoustic imaging of orthotopic brain tumors, using a 1064 nm pulsed laser as a light source.

Yin et al “Organic semiconducting polymer amphiphile for near-infrared-II light triggered phototheranostics”, Biomaterials, 2020, 232, 119684 discloses a NIR-II absorbing organic semiconducting polymer amphiphile.

“Semiconducting polymer nanoparticles for amplified photoacoustic imaging”, Nanomed Nanobiotechnol, 2018, 10 (5), 1510 is a review of the development of semiconducting nanoparticles as exogenous PA contrasts agents.

U.S. Pat. No. 10,124,111 discloses a small molecule dye for use in imaging at 1000-1700 nm wavelengths.

### SUMMARY

In some embodiments, the present disclosure provides a light-absorbing agent for use in photoacoustic imaging wherein the light-absorbing agent comprises nanoparticles comprising a light-absorbing material comprising an electron accepting unit and an electron donating unit wherein the electron-donating unit is a unit of formula (IIIa-1):

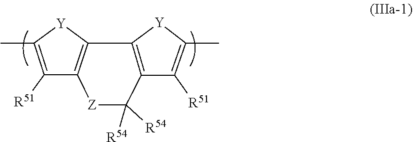

wherein:
Y in each occurrence is independently O or S;
Z in each occurrence is O, S, NR⁵⁵, or C(R⁵⁴)₂;
R⁵¹ in each occurrence is H or a substituent;
R⁵⁴ in each occurrence is independently a substituent; and
R⁵⁵ is H or a substituent.

Optionally, the light-absorbing material is a polymer; the electron-accepting unit is an electron-accepting repeat unit; and the electron donating unit is an electron-donating repeat unit. Preferably, the polymer is a conjugated polymer. Preferably, the conjugated polymer comprises alternating electron-donating and electron-accepting repeat units.

Optionally, the electron-accepting unit material comprises a group of formula (XVI):

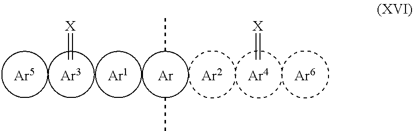

wherein Ar is a substituted or unsubstituted benzene or 6-membered heteroaromatic ring containing N and C ring atoms; Ar¹ is a substituted or unsubstituted 5- or 6-membered heteroaromatic ring containing N and C ring atoms; Ar² is a substituted or unsubstituted 5- or 6-membered heteroaromatic ring or is absent; A³ is a 5-membered ring or a substituted or unsubstituted 6-membered ring; Ar⁴ is a 5-membered ring or a substituted or unsubstituted 6-membered ring or is absent; Ar⁵ is a substituted or unsubstituted monocyclic or polycyclic group containing at least one aromatic or heteroaromatic ring; Ar⁶ is a substituted or unsubstituted monocyclic or polycyclic group containing at least one aromatic or heteroaromatic ring or is absent; and each X is independently a substituent bound to a carbon atom of Ar³ and, where present, Ar⁴ with the proviso that at least one X group is an electron withdrawing group.

Optionally, the nanoparticles comprise a core comprising the light-absorbing material and silica.

According to some embodiments, the light-absorbing material is distributed within a matrix comprising the silica. The light-absorbing material may or may not be covalently bound to the silica.

According to some embodiments, a core of the nanoparticle comprises a central region encapsulated by a shell; and the light-absorbing material is disposed in the central region. Optionally, the shell comprises or consists of the silica.

Optionally, the light-absorbing material has a peak absorption wavelength of at least 1100 nm.

Optionally, the nanoparticles have a number average diameter of less than 300 nm, optionally less than 200 nm, as determined by dynamic light scattering.

According to some embodiments, the present disclosure provides a light-absorbing agent for use in photoacoustic imaging wherein the light-absorbing agent comprises nanoparticles comprising a light-absorbing material comprising an electron accepting unit and an electron donating unit wherein the electron-accepting unit is a group of formula (XVI):

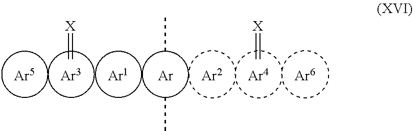

wherein Ar is a substituted or unsubstituted benzene or 6-membered heteroaromatic ring containing N and C ring atoms; Ar¹ is a substituted or unsubstituted 5- or 6-membered heteroaromatic ring containing N and C ring atoms; Ar² is a substituted or unsubstituted 5- or 6-membered heteroaromatic ring or is absent; Ar³ is a 5-membered ring or a substituted or unsubstituted 6-membered ring; Ar⁴ is a 5-membered ring or a substituted or unsubstituted 6-membered ring or is absent; Ar⁵ is a substituted or unsubstituted monocyclic or polycyclic group containing at least one aromatic or heteroaromatic ring; Ar⁶ is a substituted or unsubstituted monocyclic or polycyclic group containing at least one aromatic or heteroaromatic ring or is absent; and each X is independently a substituent bound to a carbon atom of Ar³ and, where present, Ar⁴ with the proviso that at least one X group is an electron withdrawing group.

According to some embodiments, the present disclosure provides a diagnostic agent for use in photoacoustic imaging comprising nanoparticles having a core comprising a light-absorbing material and silica.

Optionally, the light-absorbing material is distributed within a matrix comprising the silica.

In some embodiments, the light-absorbing material is covalently bound to the silica.

In some embodiments, the light-absorbing material is not covalently bound to the silica.

Optionally, the core comprises a central region encapsulated by a shell; the light-absorbing material is disposed in the central region; and the shell comprises or consists of the silica.

Optionally, the light-absorbing material is an organic light-absorbing material.

Optionally, the light-absorbing material is a conjugated organic light-absorbing material.

Optionally, the light-absorbing material is a conjugated polymer. Optionally, the conjugated polymer is a donor-acceptor polymer comprising alternating electron-donating and electron-accepting repeat units.

Optionally, the light-absorbing material has a peak absorption wavelength of at least 1100 nm.

Optionally, the nanoparticles have a number average diameter of less than 300 nm, optionally less than 200 nm, as determined by dynamic light scattering.

The present disclosure provides use of nanoparticles for the manufacture of a diagnostic agent for photoacoustic imaging (PAI) wherein the nanoparticles have a core comprising a light-absorbing material and silica.

The present disclosure provides a diagnostic method comprising irradiation of a diagnostic agent in a subject's body and measuring an acoustic response of the diagnostic agent wherein the diagnostic agent comprises nanoparticles having a core comprising a light-absorbing material and silica. In some embodiments, the present disclosure provides a diagnostic agent comprising nanoparticles comprising a light-absorbing material comprising an electron-accepting unit and an electron-donating unit wherein the electron-donating unit is a unit of formula (IIIa-1):

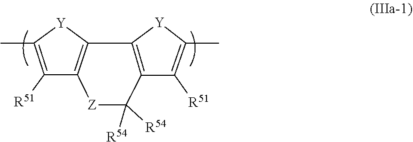

wherein:
Y in each occurrence is independently O or S;
Z in each occurrence is O, S, NR⁵⁵, or C(R⁵⁴)₂;
R⁵¹ in each occurrence is H or a substituent;
R⁵⁴ in each occurrence is independently a substituent; and
R⁵⁵ is H or a substituent.

The nanoparticles comprising the light-absorbing material comprising the unit of formula (IIIa-1) may have a core as described anywhere herein comprising silica and the light-absorbing material.

The present disclosure provides use of a light-absorbing agent for the manufacture of a diagnostic agent for photoacoustic imaging, wherein the light-absorbing agent comprises nanoparticles having a core comprising a light-absorbing material comprising an electron-accepting unit and an electron-donating unit, wherein the electron-donating unit is a unit of formula (IIIa-1):

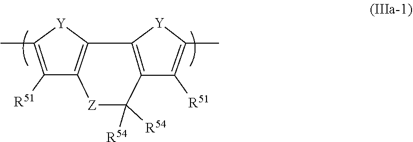

wherein:
Y in each occurrence is independently O or S;
Z in each occurrence is O, S, NR⁵⁵, or C(R⁵⁴)₂;
R⁵¹ in each occurrence is H or a substituent;
R⁵⁴ in each occurrence is independently a substituent; and
R⁵⁵ is H or a substituent.

The present disclosure provides a diagnostic method comprising irradiation of a diagnostic agent in a subject's body, and measuring an acoustic response of the light-absorbing agent, wherein the diagnostic agent comprises nanoparticles having a core comprising a light-absorbing material comprising an electron-accepting unit and an electron-donating unit wherein the electron-donating unit is a unit of formula (IIIa-1):

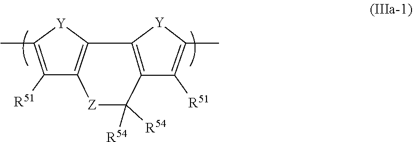

wherein:
Y in each occurrence is independently O or S;
Z in each occurrence is O, S, NR⁵⁵, or C(R⁵⁴)₂;
R⁵¹ in each occurrence is H or a substituent;
R⁵⁴ in each occurrence is independently a substituent; and
R⁵⁵ is H or a substituent.

In some embodiments, the present disclosure provides nanoparticles for use in photothermal therapy wherein the nanoparticles comprise a core comprising a light-absorbing material and silica.

In some embodiments, the present disclosure provides nanoparticles for use in photothermal therapy, wherein the nanoparticles comprise a light-absorbing material comprising an electron-accepting unit and an electron-donating unit, wherein the electron-donating unit is a unit of formula (IIIa-1):

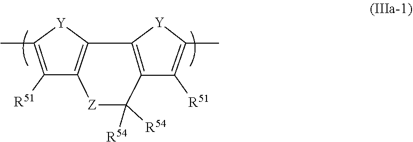

wherein:
Y in each occurrence is independently O or S;
Z in each occurrence is O, S, NR⁵⁵, or C(R⁵⁴)₂;
R⁵¹ in each occurrence is H or a substituent;
R⁵⁴ in each occurrence is independently a substituent; and
R⁵⁵ is H or a substituent.

The nanoparticles for use in photothermal therapy may be as described anywhere herein with respect to nanoparticles of a diagnostic agent.

### DESCRIPTION OF DRAWINGS

FIG. 1 is an absorption spectrum of nanoparticles in methanol for different quantities (in microlitres) of nanoparticle suspension added to methanol.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a specific atom include any isotope of that atom unless specifically stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The present inventors have found that silica nanoparticles containing a light-absorbing material may be used in photoacoustic imaging or photothermal treatment within a subject's body. By at least partly encapsulating the light-absorbing material within a silica matrix, undesirable interactions within the subject's body may be avoided, for example interactions which may limit the photoacoustic activity of the light-absorbing material or which may be harmful to the subject.

A nanoparticle as described herein may have surface groups on a core of the nanoparticle. The surface groups may include groups configured to bind to a target and/or groups selected to prevent undesirable bindings of the nanoparticles. In some embodiments, a silica nanoparticle is provided with polyethylene glycol (PEG) surface groups.

Nanoparticles comprising silica and a light-absorbing material may be formed by methods known to the skilled person, e.g., by polymerisation of a silica monomer in the presence of the light-absorbing material. Selection of the conditions for forming silica-containing nanoparticles allows for a high degree of control over the size of the resultant nanoparticles, which can be important in ensuring that the particles can be efficiently excreted by the subject they are introduced into after use.

The present inventors have further found that nanoparticles containing a donor-acceptor molecule including a donor unit of formula (IIIa-1) may provide absorption at wavelengths of at least 1100 nm, optionally in the range of 1100-1700 nm. Such nanoparticles may be particularly suitable for deep tissue PAI.

Unless stated otherwise, absorption spectra of light-absorbing materials as described herein are measured in solution, e.g. toluene, water or methanol solution, using a Cary 5000 UV-VIS-NIR Spectrometer. Measurements are taken from 175 nm to 3300 nm using a PbSmart NIR detector for extended photometric range with variable slit widths (down to 0.01 nm) for optimum control over data resolution. A baseline run with water in front and back 5 ml matched cuvettes (600 to 250 nm) following which the back cuvette reference remains as water and the front cuvette is changed to a sample of 1 mg/ml diluted 1 in 100 for a dissolved light-absorbing material.

### Light-Absorbing Molecule

The light-absorbing material is preferably an organic light-absorbing material. An organic light-absorbing material as described herein may be a non-polymeric (small molecule) or polymeric material. Optionally, a non-polymeric light-absorbing material comprises one of an anionic and cationic group and the nanoparticle comprises a polymer having a repeat unit comprising the other of an anionic and cationic group. Ionic bonding between the polymer and non-polymeric light-absorbing material may prevent leaching of the non-polymeric light-absorbing material from the particle.

Preferably, a non-polymeric material as described herein has a molecular weight of less than 5,000 Daltons, optionally less than 3,000 Daltons.

Preferably, the polystyrene-equivalent number-average molecular weight (Mn) measured by gel permeation chromatography of a polymeric material as described herein is in the range of about 5×10³ to 1×10⁸, and preferably 1×10⁴ to 5×10⁶. The polystyrene-equivalent weight-average molecular weight (Mw) of a polymeric material as described herein is preferably 3×10³ to 1×10⁸, and preferably 1×10⁴ to 1×10⁷.

The organic light-absorbing material is preferably a conjugated organic light-absorbing material, more preferably a conjugated light-absorbing polymer. A conjugated material as described herein comprises at least two units which are directly conjugated to one another, e.g., an electron-accepting unit directly conjugated to an electron-donating unit.

The conjugated organic light-absorbing material preferably comprises an electron-donating unit D and an electron-accepting unit A. In the case of a conjugated organic light-absorbing polymer, the electron-donating unit D and the electron-accepting unit A are, respectively, an electron-donating repeat unit and an electron-accepting repeat unit.

By “conjugated polymer” as used herein is meant a polymer having a backbone containing repeat units that are directly conjugated to adjacent repeat units in the polymer backbone, e.g., an electron-accepting repeat unit directly conjugated to an electron-donating repeat unit.

The, or each, electron-accepting unit D has a LUMO level that is deeper (i.e., further from vacuum) than the, or each, electron-donating unit A, preferably at least 1 eV deeper. The LUMO levels of an electron-donating unit and an electron-accepting unit A may be modelled using Gaussian09 software available from Gaussian using Gaussian09 with B3LYP (functional).

A non-polymeric conjugated organic light-absorbing compound may be selected from, without limitation, formulae (Ia), (Ib) or (Ic).

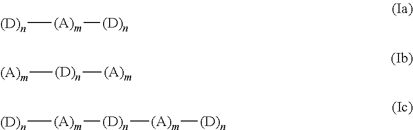

wherein each n is at least 1 and each m is at least 1.

If n is greater than 1 then D in each occurrence may be the same or different.

If m is greater than 1 then A in each occurrence may be the same or different.

A conjugated organic light-absorbing polymer is preferably a donor-acceptor polymer having a repeating structure of formula (II):

-[(D)n-(A)m]- (II)

In a preferred embodiment of any of formulae (Ia), (Ib), (Ic) and (II), n is 1 and m is 1.

In another preferred embodiment of any of formulae (Ia), (Ib), (Ic) and (II), n is greater than 1, preferably 2 or 3, and m is 1.

Each unit D and each unit A is optionally unsubstituted or substituted with one or more substituents. The substituents may be selected according to a desired solubility of the light-absorbing material.

Optionally, electron-donating units D are selected from formulae (IIIa)-(IIIq):

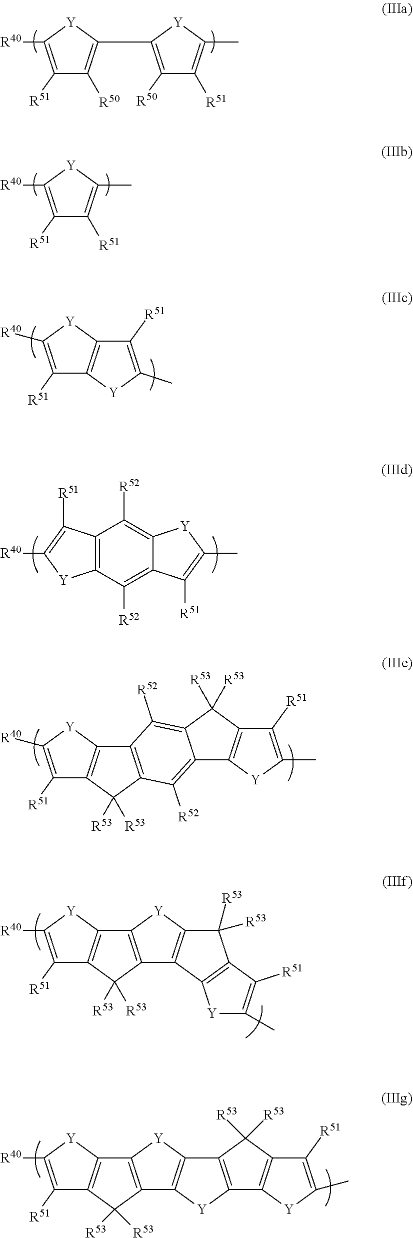

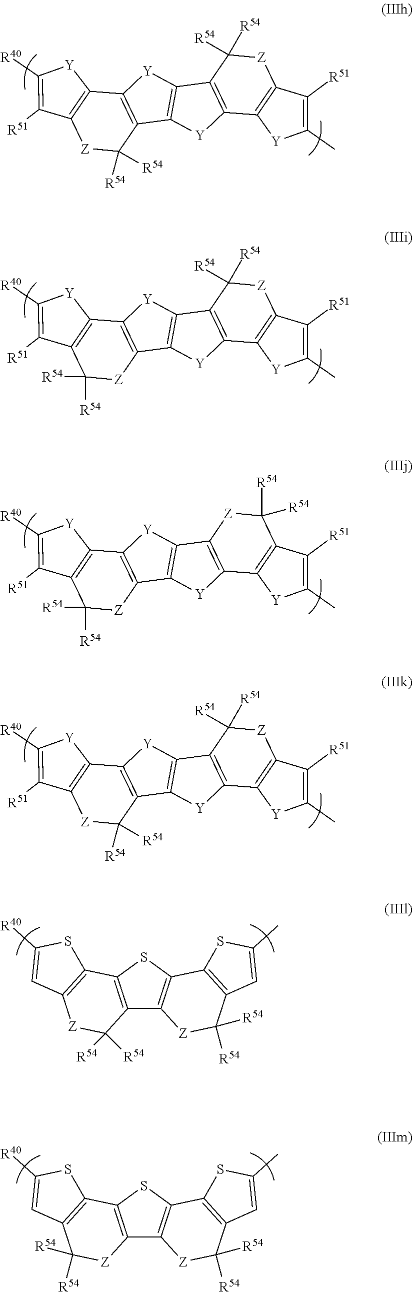

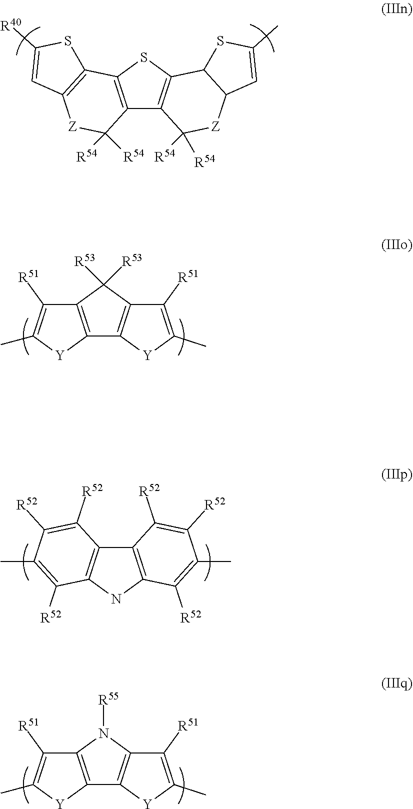

wherein Y in each occurrence is independently O or S, preferably S;
Z in each occurrence is O, S, NR⁵⁵, or C(R⁵⁴)₂;
R⁴⁰ is H or a substituent in the case where D is a monovalent unit, e.g., a monovalent unit D of formula (Ia) or (Ic) or R⁴⁰ is a direct bond in the case where D is a divalent unit, e.g., a divalent unit D of formula (Ib), (Ic) or (II);
R⁵⁰, R⁵¹, R⁵², R⁵⁴ and R⁵⁵ independently in each occurrence is H or a substituent wherein R⁵⁰ groups may be linked to form a ring; and R⁵³ independently in each occurrence is a substituent.

Substituents R⁴⁰, R50, R51, R⁵², R⁵³, R⁵⁴ and R⁵⁵ may each independently be an ionic or non-ionic substituent.

An ionic substituent may have formula (IV):

-(Sp)p-(R⁴)q (IV)
wherein Sp is a spacer group; R⁴ is an ionic group; p is 0 or 1; q is 1 if p is 0; and q is at least 1, optionally 1, 2 or 3, if p is 1.

R⁴ may be an anionic or cationic group. Exemplary anionic groups are —COO⁻, a sulfonate group; hydroxide; sulfate; phosphate; phosphinate; or phosphonate. An exemplary cationic group is —N(R⁵)₃⁺ wherein R⁵ in each occurrence is H or C₁₋₂₀ hydrocarbyl. Preferably, each R⁵ is a C₁₋₂₀ hydrocarbyl.

A C₁₋₂₀ hydrocarbyl as described anywhere herein is preferably selected from C₁₋₂₀ alkyl; unsubstituted phenyl; and phenyl substituted with one or more C₁₋₁₂ alkyl groups.

Optionally, Sp is selected from:
C₁₋₂₀ alkylene or phenylene-C₁₋₂₀ alkylene wherein one or more non-adjacent C atoms may be replaced with O, S, N or C═O;
a C₆₋₂₀ arylene or 5-20 membered heteroarylene, more preferably phenylene, which, in addition to the one or more substituents R⁴, may be unsubstituted or substituted with one or more non-ionic substituents, optionally one or more C₁₋₂₀ alkyl groups wherein one or more non-adjacent C atoms may be replace with O, S, N or C═O.

In a preferred embodiment, Sp¹ is a C₆₋₂₀ arylene or 5-20 membered heteroarylene, more preferably phenylene, substituted with at least one group of formula:

—O(R⁶O)ᵥ—R⁷
wherein R⁶ in each occurrence is a C₁₋₁₀ alkylene group; R⁷ is H or C₁₋₅ alkyl, and v is 0 or a positive integer, optionally 1-10. Preferably, v is at least 2. More preferably, v is 2 to 5. The value of v may be the same in all groups of formula —O(R⁶O)ᵥ—R⁷. The value of v may differ between different groups of formula —O(R⁶O)ᵥ—R⁷ of the same light-absorbing material.

If a C atom of an alkyl group as described anywhere herein is replaced with another atom or group, the replaced C atom may be a terminal C atom of the alkyl group or a non-terminal C-atom.

By “non-terminal C atom” of an alkyl group as used anywhere herein means a C atom other than the C atom of the methyl group at the end of an n-alkyl chain or the C atoms of the methyl groups at the ends of a branched alkyl chain.

If a terminal C atom of a group as described anywhere herein is replaced then the resulting group may be an anionic group comprising a countercation, e.g. an ammonium or metal countercation, preferably an ammonium or alkali metal cation.

A light-absorbing material as described herein comprising cationic or anionic groups comprises counterions to balance the charge of these ionic groups. An anionic or cationic group and counterion may have the same valency, with a counterion balancing the charge of each anionic or cationic group. The anionic or cationic group may be monovalent or polyvalent. Preferably, the anionic and cationic groups are monovalent.

The light-absorbing material may comprise a plurality of anionic or cationic polar groups wherein the charge of two or more anionic or cationic groups is balanced by a single counterion.

In the case of an anionic group, the cation counterion is optionally a metal cation, optionally Li⁺, Na⁺, K⁺, Cs⁺, preferably Cs⁺, or an organic cation, optionally ammonium, such as tetraalkylammonium, ethylmethyl imidazolium or pyridinium.

In the case of a cationic group, the anion counterion is optionally a halide; a sulfonate group, optionally mesylate or tosylate; hydroxide; carboxylate; sulfate; phosphate; phosphinate; phosphonate; or borate.

Non-ionic substituents R⁴⁰, R⁵⁰, R⁵¹, R⁵², R⁵³, R⁵⁴ and R⁵⁵ are preferably selected from the group consisting of:
linear, branched or cyclic C₁₋₂₀ alkyl wherein one or more non-adjacent, non-terminal C atoms may be replaced by O, S, NR⁸, CO or COO wherein R⁸ is a C₁₋₂₀ hydrocarbyl and one or more H atoms of the C₁₋₂₀ alkyl may be replaced with F; and
a group of formula (Ak)u-(Ar¹⁰)v wherein Ak is a C₁₋₁₂ alkylene chain in which one or more C atoms may be replaced with O, S, CO or COO; u is 0 or 1; Ar¹⁰ in each occurrence is independently an aromatic or heteroaromatic group which is unsubstituted or substituted with one or more substituents; and v is at least 1.

Ar⁴ is preferably an aromatic group, more preferably phenyl, which may be unsubstituted or substituted with one or more substituents selected from ionic groups, optionally ionic groups of formula R⁴ as described above; and non-ionic substituents selected from F; CN; NO₂; and C₁₋₂₀ alkyl wherein one or more non-adjacent, non-terminal C atoms may be replaced with O, S, COO or NR⁸.

Substituents R⁴⁰, R⁵⁰, R⁵¹, R⁵², R⁵³, R⁵⁴ and R⁵⁵ may be selected according to the desired solubility of the light-absorbing material in a solvent.

One or more of R⁴⁰, R⁵⁰, R⁵¹, R⁵², R⁵³, R⁵⁴ and R⁵⁵ may be an ionic or a non-ionic polar substituent in order to enhance solubility in a polar solvent, e.g., an alcohol or water.

One or more of R⁴⁰, R⁵⁰, R⁵¹, R⁵², R⁵³, R⁵⁴ and R⁵⁵ may be a non-polar substituent in order to enhance solubility in a non-polar solvent, e.g. benzene with one or more substituents selected from C₁₋₁₂ alkyl and alkoxy groups.

Preferred non-polar substituents are C₁₋₂₀ hydrocarbyl groups as described herein.

Preferred polar non-ionic substituents as described herein are C₁₋₂₀ alkyl wherein one or more non-adjacent C atoms are replaced by O, S, NR⁸, CO or COO, more preferably substituents comprising or consisting of a group of formula —O(CH₂CH₂O)ₜR⁹ wherein t is at least 1, optionally 1-10, R⁸ is as described above and R⁹ is a C₁₋₅ alkyl group, preferably methyl.

Preferably, each R⁵¹ is H.

A preferred group R⁵⁴ is phenyl which is unsubstituted or substituted with one or more groups R⁷⁰ wherein R⁷⁰ in each occurrence is a substituent, preferably a substituent selected from C₁₋₃₀ alkyl wherein one or more non-adjacent, non-terminal C atoms may be replaced with O, S, COO or NR⁸, for example a group selected from —(OCH₂CH₂)r-OAk¹ and —(OCH₂CH₂)r-NAk¹₂ wherein r is 1-10 and Ak¹ is C₁₋₄ alkyl.

In a preferred embodiment, the unit of formula (IIIa) has formula (IIIa-1) in which the R⁵⁰ groups are linked to form a group of formula —Z—C(R⁵⁴)₂— wherein Z is O, S NR⁵⁵, or C(R⁵⁴)₂:

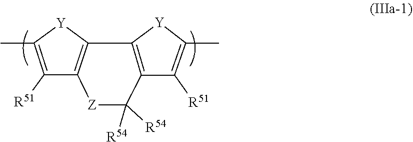

In some embodiments, n of formula (Ia), (Ib), (Ic) and (II) is more than 1 and the D units are one of formulae (IIIa)-(IIIq).

In these embodiments, each of the n units may be connected in any orientation. For example, in the case where each D of -(D)n- is a group of formula (IIIa-1) and n is 2, -(D)n- may be selected from any of:

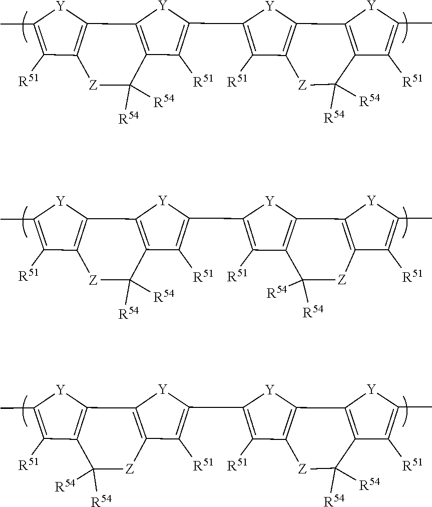

wherein Y in each occurrence, Z in each occurrence; R⁵¹ in each occurrence and R⁵⁴ in each occurrence are independently the same or different.

In some embodiments, n of formula (Ia), (Ib), (Ic) or (II) is more than 1; -(D)n- includes a first electron-donating unit which is one of formulae (IIIa)-(IIIq); and -(D)n- includes a second electron-donating unit D selected from another of formulae (IIIa)-(IIIq).

Optionally, the first electron-donating unit has formula (IIIa-1) and the second electron-donating unit is selected from (IIIb) and (IIIc). Optionally, according to these embodiments, the second electron-donating unit D of formula (Ia), (Ib), (Ic) or (II) is disposed between the first donor group D and the electron-accepting unit or units A.

An exemplary group of formula -(D)n- containing first and second donor units D is:

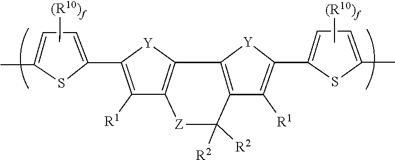

wherein R¹⁰ in each occurrence is a substituent, preferably a substituent as described with respect to R⁴⁰, R⁵⁰, R⁵¹, R⁵², R⁵³, R⁵⁴ and R⁵⁵, and R⁵⁵, each occurrence is 0, 1 or 2.

In the case where electron-accepting unit A of the light-absorbing material is a monovalent unit, it may be selected from formulae:

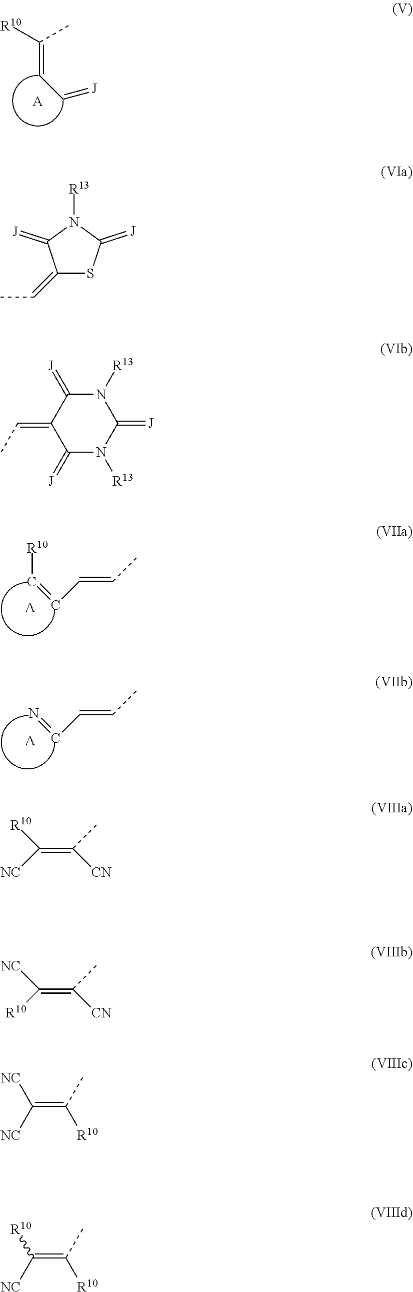

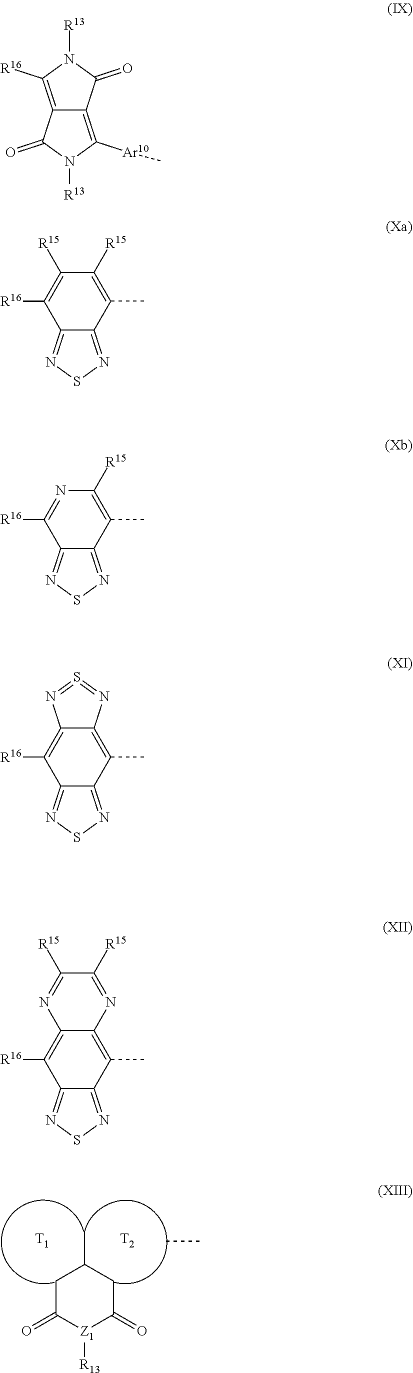

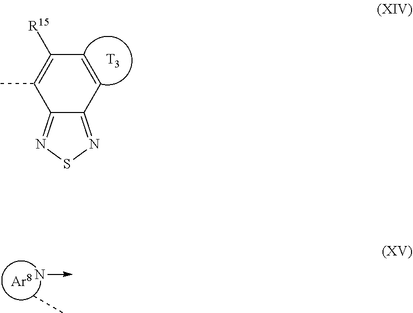

wherein [symbol] represents to a bond an electron-donating unit D.

A is a 5- or 6-membered ring which is unsubstituted or substituted with one or more substituents and which may be fused to one or more further rings.

R¹⁰ is H or an ionic or non-ionic substituent as described herein, preferably a substituent selected from the group consisting of C₁₋₁₂ alkyl wherein one or more non-adjacent C atoms may be replaced with O, S, COO or CO and one or more H atoms of the alkyl may be replaced with F; and an aromatic group Ar⁷, optionally phenyl, which is unsubstituted or substituted with one or more substituents selected from F and C₁₋₁₂ alkyl wherein one or more non-adjacent C atoms may be replaced with O, S, COO or CO.

Preferably, R¹⁰ is H.

J is O or S.

R¹³ in each occurrence is a substituent, optionally C₁₋₁₂ alkyl wherein one or more non-adjacent C atoms may be replaced with O, S, COO or CO and one or more H atoms of the alkyl may be replaced with F.

R¹⁵ in each occurrence is independently H or an ionic or non-ionic substituent, optionally H or a substituent selected from:
F;
CN;
NO₂;
C₁₋₁₂ alkyl wherein one or more non-adjacent C atoms may be replaced with O, S, COO or CO and one or more H atoms of the alkyl may be replaced with F;
an aromatic group Ar⁷, optionally phenyl, which is unsubstituted or substituted with one or more substituents selected from F, CN, NO₂ and C₁₋₂₀ alkyl, preferably C₁₋₁₂ alkyl wherein one or more non-adjacent C atoms may be replaced with O, S, COO or CO; or

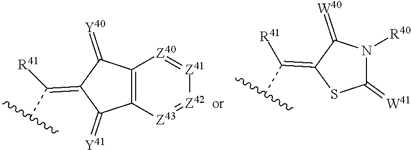

wherein Z⁴⁰, Z⁴¹, Z⁴² and Z⁴³ are each independently CR¹³ or N wherein R¹³ in each occurrence is H or a substituent, preferably a C₁₋₂₀ hydrocarbyl group;
Y⁴⁰ and Y⁴¹ are each independently O, S, NX⁷⁰ wherein X⁷⁰ is C₁₋₂₀ alkyl, CN or COOR⁴¹; or CX⁶⁰X⁶¹ wherein X⁶⁰ or X⁶¹ is independently CN, CF₃ or COOR⁴¹;
W⁴⁰ and W⁴¹ are each independently O, S, NX⁷⁰ wherein X⁷⁰ is C₁₋₂₀ alkyl, CN or COOR⁴¹; or CX⁶⁰X⁶¹ wherein X⁶⁰ or X⁶¹ is independently CN, CF₃ or COOR⁴¹; and
R⁴¹ in each occurrence is H or a substituent, preferably H or a C₁₋₂₀ hydrocarbyl group.
R¹⁶ is H or a substituent, preferably a substituent selected from:
—(Ar⁹)_{w}, wherein Ar⁹ in each occurrence is independently an unsubstituted or substituted aryl or heteroaryl group, preferably thiophene, and w is 1, 2 or 3;

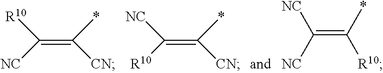

and
C₁₋₁₂ alkyl wherein one or more non-adjacent C atoms may be replaced with O, S, COO or CO and one or more H atoms of the alkyl may be replaced with F.

Substituents of Ar⁹, where present, are optionally selected from C₁₋₁₂ alkyl wherein one or more non-adjacent C atoms may be replaced with O, S, COO or CO and one or more H atoms of the alkyl may be replaced with F.

Z¹ is N or P

T¹, T² and T³ each independently represent an aryl or a heteroaryl ring which may be fused to one or more further rings. Substituents of T¹, T² and T³, where present, are optionally selected from non-H groups of R¹⁵.

Ar⁸ is a fused heteroaromatic group which is unsubstituted or substituted with one or more non-H substituents R¹⁰.

A preferred unit of formula (V) is formula (Va):

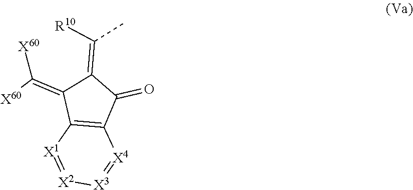

wherein:
R¹⁰ is as described above;
[symbol] represents a linking position to an electron-donating group;
each X¹—X⁴ is independently CR¹⁷ or N wherein R¹⁷ in each occurrence is H or a substituent selected from C₁₋₂₀ hydrocarbyl and an electron withdrawing group; and
X⁶⁰ in each occurrence is independently CN, CF₃ or COOR⁴¹ wherein R⁴¹ in each occurrence is H or a substituent, preferably H or a C₁₋₂₀ hydrocarbyl group.

Optionally, the electron withdrawing group R¹⁷ is F, Cl, Br or CN.

Preferably, each X⁶⁰ is CN.

Exemplary units of formula (VIIa) or (VIIb) include:

Exemplary groups of formula (VIIa) include:

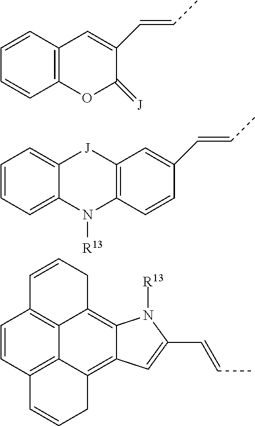

Exemplary groups of formula (VIIb) include:

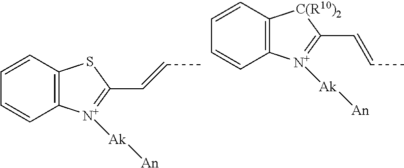

An exemplary group of formula (VIId) is:

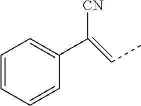

An exemplary group of formula (VIIIb) is:

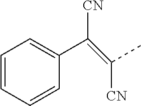

An exemplary group of formula (Xa) is:

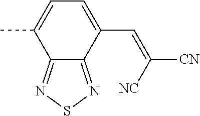

wherein Ak is a C₁₋₁₂ alkylene chain in which one or more C atoms may be replaced with O, S, CO or COO; An is an anion, optionally —SO₃⁻; and each benzene ring is independently unsubstituted or substituted with one or more substituents selected from substituents described with reference to R¹⁰.

Exemplary unit of formula (XIII) are:

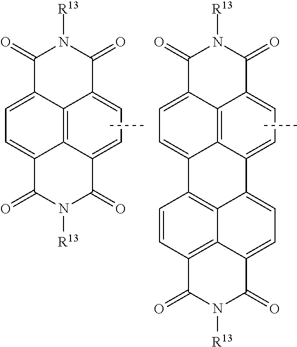

An exemplary unit of formula (XIV) is:

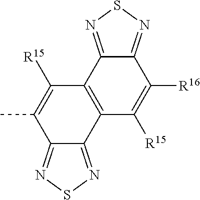

In the case where at least one electron-accepting unit is a group of formula (XV), an adjacent electron-donating unit is substituted with —B(R¹⁴)₂ wherein R¹⁴ in each occurrence is a substituent, optionally a C₁₋₂₀ hydrocarbyl group; [symbol] is bound to the electron-donating unit; and →is a bond to the boron atom of —B(R¹⁴)₂.

The electron-donating unit, the unit of formula (XV) and the B(R¹⁴)₂ substituent may be linked together to form a 5- or 6-membered ring.

In some embodiments, the electron-accepting unit of formula (XV) is selected from formulae (XVa), (XVb) and (XVc):

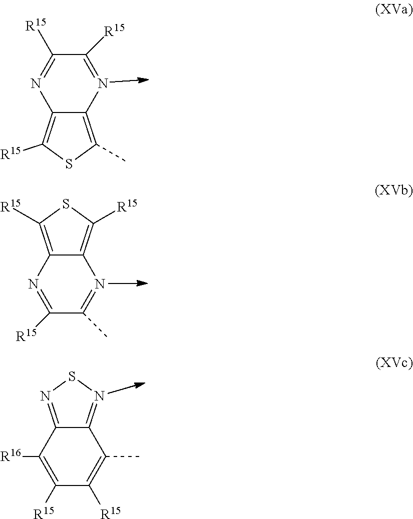

In some embodiments, divalent electron-accepting units are preferably selected from
divalent analogues of formulae (IX)-(XII) wherein R¹⁶ is a bond to an electron-donating unit;
divalent analogues (XIIIa) and (XIVa) of formulae (XIII) and (XIV), respectively:

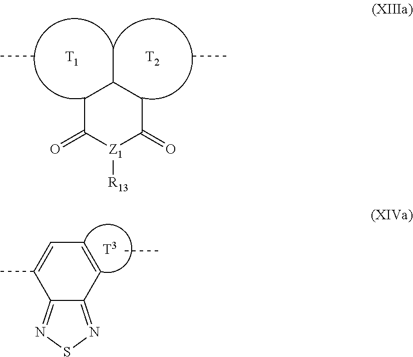

Most preferably, divalent electron-accepting units have formula (XVI):

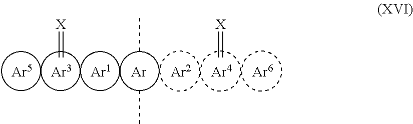

wherein Ar is a substituted or unsubstituted benzene or 6-membered heteroaromatic ring containing N and C ring atoms; Ar¹ is a substituted or unsubstituted 5- or 6-membered heteroaromatic ring containing N and C ring atoms; Ar² is a substituted or unsubstituted 5- or 6-membered heteroaromatic ring or is absent; Ar³ is a 5-membered ring or a substituted or unsubstituted 6-membered ring; Ar⁴ is a 5-membered ring or a substituted or unsubstituted 6-membered ring or is absent; Ar⁵ is a substituted or unsubstituted monocyclic or polycyclic group containing at least one aromatic or heteroaromatic ring; Ar⁶ is a substituted or unsubstituted monocyclic or polycyclic group containing at least one aromatic or heteroaromatic ring or is absent; and each X is independently a substituent bound to a carbon atom of Ar³ and, where present, Ar⁴ with the proviso that at least one X group is an electron withdrawing group.

It will be understood that the possibility of substituting A¹, Ar², Ar⁵ and Ar⁶ will be dependent on the structure of formula (I) and the availability of substitution positions. For example, if Ar² is present and is a 6-membered aromatic or heteroaromatic ring containing less than four heteroatoms in the ring, then substitution may be present; if Ar² is a 5-membered heteroaromatic ring, containing less than three heteroatoms in the ring, then substitution may be present; if Ar⁵ is a monocyclic or polycyclic group containing a least one aromatic ring, then substitution may be present.

Substituents of Ar¹, Ar², Ar⁵ and Ar⁶, where present, are preferably selected from substituents R⁶¹ wherein R⁶¹ in each occurrence is selected from the group consisting of:
F;
Cl;
CN;
NO₂;
linear, branched or cyclic C₁₋₃₀ alkyl wherein one or more non-adjacent C atoms may be replaced by O, S, NR⁷, CO or COO wherein R⁷ is a C₁₋₁₂ hydrocarbyl and one or more H atoms of the C₁₋₂₀ alkyl may be replaced with F; and
a group of formula (Ak)u-(Ar⁷)v wherein Ak is a C₁₋₁₂ alkylene chain in which one or more non-adjacent C atoms may be replaced with O, S, CO or COO; u is 0 or 1; Ar⁷ in each occurrence is independently an aromatic or heteroaromatic group which is unsubstituted or substituted with one or more substituents; and v is at least 1, optionally 1, 2 or 3.

Substituents of Ar⁷, if present, are preferably selected from F; Cl; NO₂; CN; and C₁₋₂₀ alkyl wherein one or more non-adjacent C atoms may be replaced with O, S, CO or COO. Preferably, Ar⁷ is phenyl.

More preferred substituents R⁶¹ are F; Cl; C₁₋₂₀ alkyl wherein one or more H atoms may be replaced with F and one or more C atoms may be replaced with O, S or COO; and phenyl which is unsubstituted or substituted with one or more substituents selected from F, CN and C₁₋₃₀ alkyl wherein one or more H atoms of the C₁₋₃₀ alkyl may be replaced with F and one or more C atoms of the C₁₋₃₀ alkyl may be replaced with O, S or COO. Exemplary substituents R⁶¹ include —(OCH₂CH₂)r-OAk¹ and —(OCH₂CH₂)r-NAk¹₂ wherein r is 1-10 and Ak¹ is C₁₋₄ alkyl

Preferably, the unit of formula (XVI) is selected from formulae (XVI-1)-(XVI-31):

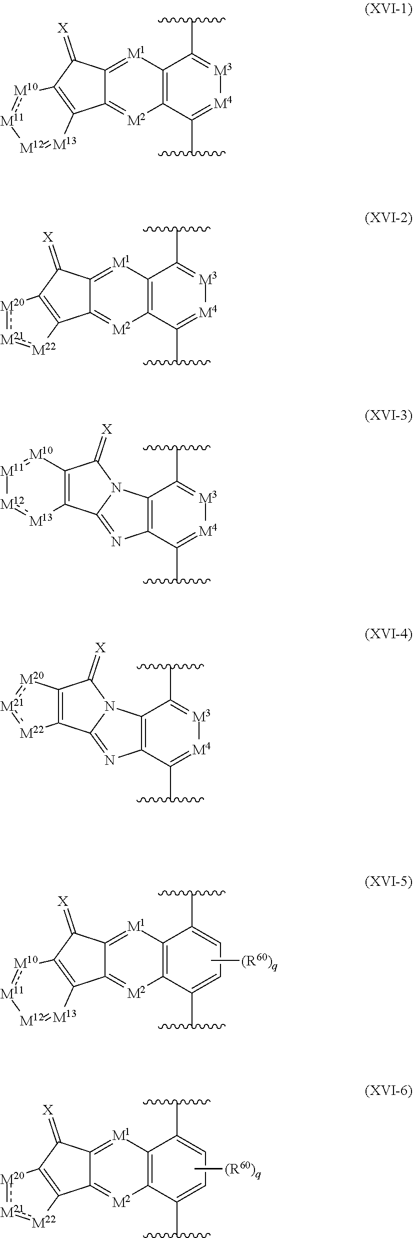

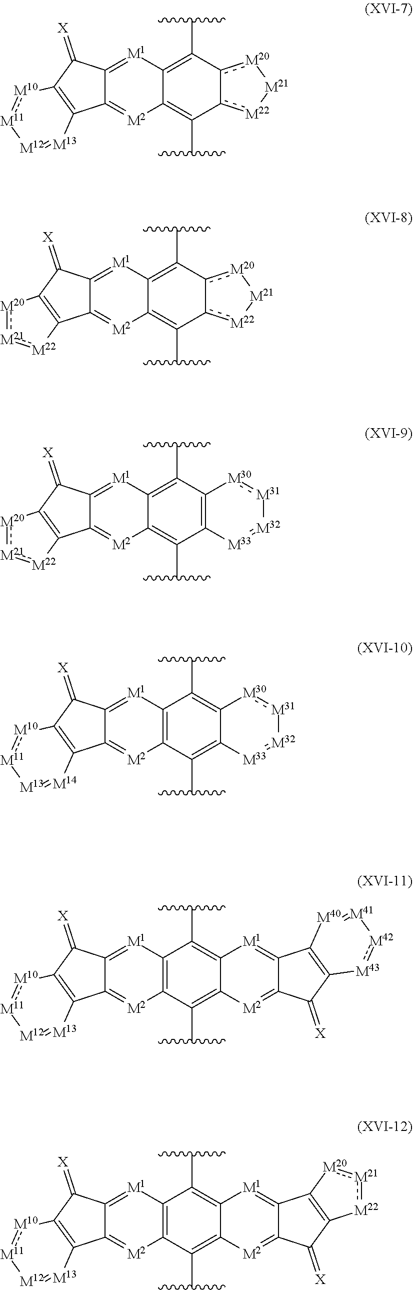

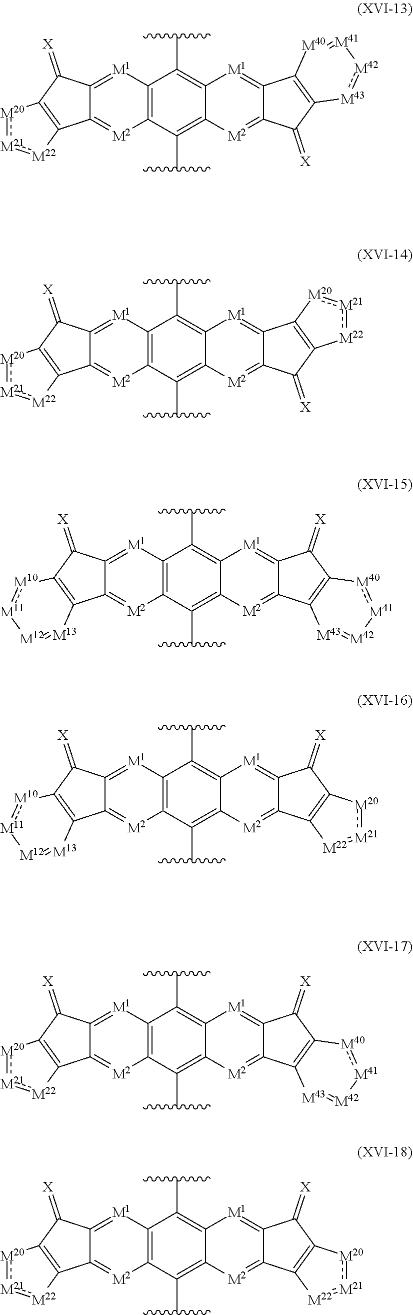

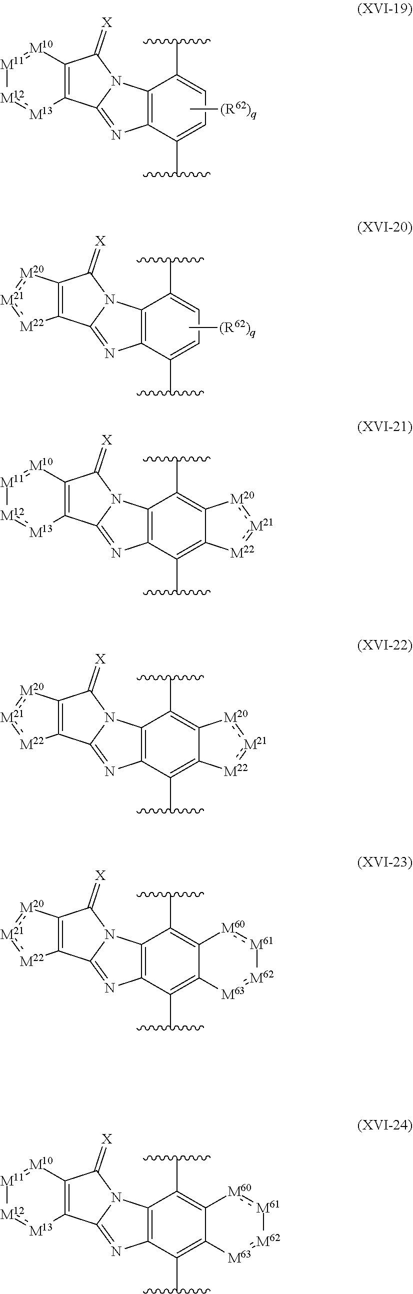

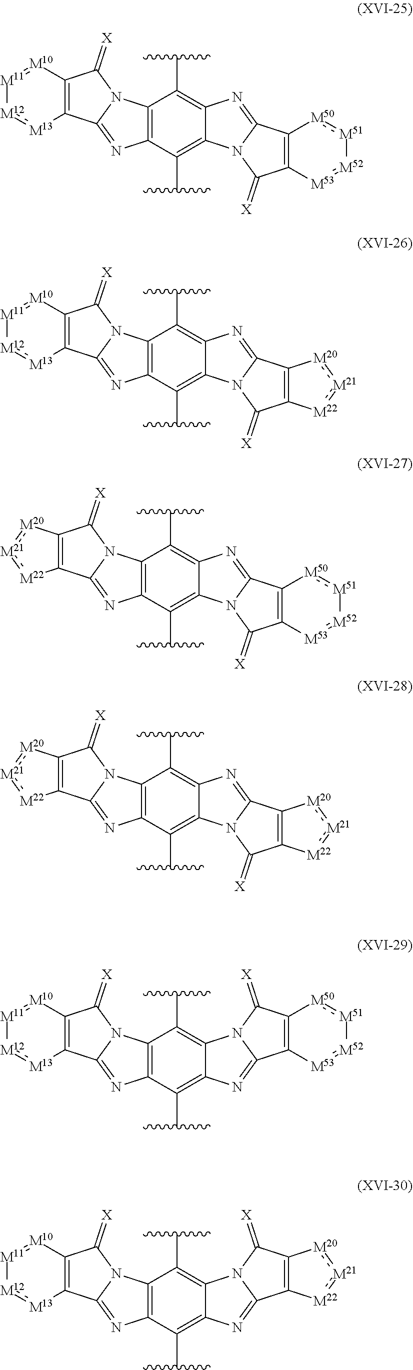

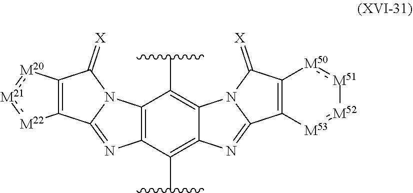

wherein
   M¹, M², M³ and M⁴, is independently CR⁶¹ or N wherein R⁶¹ in each occurrence is H or a substituent as described above;
   M¹⁰, M¹¹, M¹², M¹³, M²⁰, M²¹, M²², M³⁰, M³¹, M³², M³³, M⁴⁰, M⁴¹, M⁴², M⁴³, M⁵⁰, M⁵¹, M⁵², and M⁵³ is independently N, S, O or CR⁶¹, wherein R⁶¹ in each occurrence is a H or a substituent and with the proviso that a S or O is not adjacent to another S or O;
   X is independently an electron withdrawing group;
   R⁶² is a substituent; and
   q is 0, 1, 2, 3 or 4.

Preferably, each unit of formula (XVI) is bound directly to at least one electron-donating unit D.

Preferably, Ar is benzene or a 5- or 6-membered heteroaromatic ring consisting of N and C ring atoms.

Preferably no more than 2 ring atoms of Ar are N atoms.

Preferably, Ar is selected from benzene, pyridine, and pyridazine.

More preferably, Ar is selected from benzene and pyridine.

Preferably, Ar¹ is a substituted or unsubstituted 5- or 6-membered heteroaromatic ring consisting of N and C ring atoms; consisting of N, C and O ring atoms; or consisting of N, C and S ring atoms.

Preferably no more than 2 ring atoms of Ar¹ are N atoms.

Optionally, no more than 1 ring atom of Ar¹ is an O or S atom.

Preferably, Ar¹ is selected from imidazole, pyridine, thiazine, pyrazine, and oxazine.

More preferably, Ar¹ is selected from imidazole and pyrazine.

Preferably, Ar2 where present is as described for Ar¹ with the proviso that when Ar² is a 5-membered ring, Ar² is selected from imidazole and thiadiazole.

Preferably Ar³ is a 5-membered carbocyclic ring.

Preferably Ar⁴, where present is a 5-membered carbocyclic ring.

Preferably, Ar⁵ is independently a substituted or unsubstituted monocyclic or polycyclic group containing at least one aromatic or heteroaromatic ring, wherein the heteroaromatic ring consists of N and C ring atoms; consists of N, C and S ring atoms; or consists of N, C and O ring atoms.

Preferably no more than 2 ring atoms of Ar⁵ are N atoms.

Optionally, no more than 1 ring atom Ar⁵ is an O or S atom.

Preferably, Ar⁵ is selected from benzene, pyrrole, pyrazole, imidazole, oxazole, thiazole, pyridine, thiazine, a diazine including pyrimidine, pyridazine, pyrazine, thiadiazole, oxadiazole, oxazine, and triazole.

Preferably, Ar⁵ is selected from benzene, thiadiazole, triazole and a diazine for example pyrimidine, pyridazine or pyrazine.

More preferably, Ar⁵ is benzene.

Preferably, Ar⁶ where present is selected from groups as defined for Ar⁵.

Optionally, each R⁶⁰ and R⁶² is independently selected from H or a substituent described with respect to R⁶¹. Preferably each R⁶⁰ and R⁶² is independently selected from H, F, Cl, CN; C₁₋₂₀ alkyl wherein one or more H atoms may be replaced by F; unsubstituted phenyl; or phenyl substituted with one or more substituents selected from F and C₁₋₂₀ alkyl groups wherein one or more H atoms may be replaced with F and one or more C atoms may be replaced with O, S or COO.

Preferably, each electron-withdrawing group X is independently selected from O, S and NX⁷⁰ wherein X⁷⁰ is C₁₋₂₀ alkyl, CN or COOR⁸⁰; and CX¹⁰X¹¹ wherein X¹⁰ and X¹¹ are each independently F, Cl, Br. CN, NO₂, CF₃, or COOR⁸⁰, wherein R⁸⁰ is H or a substituent, preferably H or a C₁₋₂₀ hydrocarbyl group, and preferably each of X¹⁰ and X¹¹ is F or CN.

Optionally, each electron-withdrawing group is NX⁷⁰, wherein X⁷⁰ is selected from C₁₋₂₀ alkyl wherein one or more non-adjacent C atoms may be replaced with O, S, COO or CO and one or more H atoms of the alkyl may be replaced with F; phenyl which is unsubstituted or substituted with one or more substituents, optionally one or more C₁₋₁₂ alkyl groups wherein one or more non-adjacent C atoms may be replaced with O, S, COO or CO and one or more H atoms of the alkyl may be replaced with F; and a heteroaromatic group which is unsubstituted or substituted with one or more substituents.

Preferably, each electron-withdrawing group X is independently selected from O, S and NX⁷⁰ wherein X⁷⁰ is C₁₋₂₀ alkyl, CN, COOR⁸⁰; C₁ to C₂₀ alkyl chain where any C can be replaced by O or S, substituted or unsubstituted 5- or 6-membered aromatic or heteroaromatic ring; and CX¹⁰X¹¹ wherein X¹⁰ and X¹¹ are each independently selected from F, Cl, Br. CN, NO₂, CF₃, and COOR⁸⁰, wherein R⁸⁰ is H or a substituent, preferably a C₁₋₂₀ hydrocarbyl group, and preferably each of X¹⁰ and X¹¹ is F.

Preferably, each electron-withdrawing group X is independently selected from O and CX¹⁰X¹¹ wherein X¹⁰ and X¹¹ are each independently CN or COOR⁸⁰.

More preferably, each electron-withdrawing group X is independently selected from O and CX¹⁰X¹¹ wherein X¹⁰ and X¹¹ are each CN.

In a preferred embodiment, Ar is an optionally substituted benzene or a 6-membered heteroaromatic ring containing N and C atoms; Ar¹ is a 5- or 6-membered heteroaromatic ring containing N and C atoms; Ar² is an optionally substituted 5- or 6-membered heteroaromatic ring or is absent; Ar³ is a 5- or 6-membered ring; Ar⁴ is a 5- or 6-membered ring or is absent; Ar⁵ is an optionally substituted monocyclic or polycyclic group containing at least one aromatic or heteroaromatic ring; Ar⁶ is an optionally substituted monocyclic or polycyclic group containing at least one aromatic or heteroaromatic ring or is absent; and each X is independently an electron withdrawing group, bound to the C atoms of Ar³ and Ar⁴; and wherein the material further comprises a conjugated electron-donating unit D of formula (II).

In a more preferred embodiment, Ar is benzene; Ar¹ is a 6-membered heteroaromatic ring containing N and C atoms; Ar² is a substituted 6-membered heteroaromatic ring; Ar³ is a 5-membered ring; Ar⁵ is monocyclic containing one aromatic ring; and X is an electron withdrawing group bound to the C atom of Ar³; and wherein the material further comprises a conjugated electron-donating unit D of formula (II).

In a preferred embodiment, Ar is benzene; Ar¹ is a 6-membered heteroaromatic ring containing N and C atoms; Ar² is an optionally substituted 5- or 6-membered heteroaromatic ring or is absent; Ar³ is a 5-membered ring; Ar⁴ is a 5-membered ring or is absent; Ar⁵ is an optionally substituted monocyclic or polycyclic group containing at least one aromatic or heteroaromatic ring; Ar⁶ is independently an optionally substituted monocyclic or polycyclic group containing at least one aromatic or heteroaromatic ring or is absent; and each X is independently an electron withdrawing group bound to the C atoms of Ar³ and/or Ar⁴.

In a preferred embodiment, Ar is benzene; Ar¹ is a 6-membered heteroaromatic ring containing N and C atoms; Ar² 5-membered heteroaromatic ring; Ar³ is a 5-membered ring; Ar⁵ is monocyclic group containing one aromatic ring; and X an electron withdrawing group bound to the C atom of Ar³.

In a preferred embodiment, Ar is benzene; Ar¹ is a 5-membered heteroaromatic ring containing N and C atoms; Ar² is an optionally substituted 5- or 6-membered heteroaromatic ring or is absent; Ar³ is a 5-membered ring; Ar⁴ is a 5-membered ring or is absent; Ar⁵ is an optionally substituted monocyclic or polycyclic group containing at least one aromatic or heteroaromatic ring; Ar⁶ is an optionally substituted monocyclic or polycyclic group containing at least one aromatic or heteroaromatic ring or is absent; and each X is independently an electron withdrawing group bound to the C atoms of Ar³ and/or Ar⁴.

Exemplary units of formula (XVI) include the following which may be unsubstituted or substituted with one or more substituents R⁶¹ as described above:

Preferred divalent electron-accepting groups, e.g., electron-accepting repeat units of a donor-acceptor polymer are:

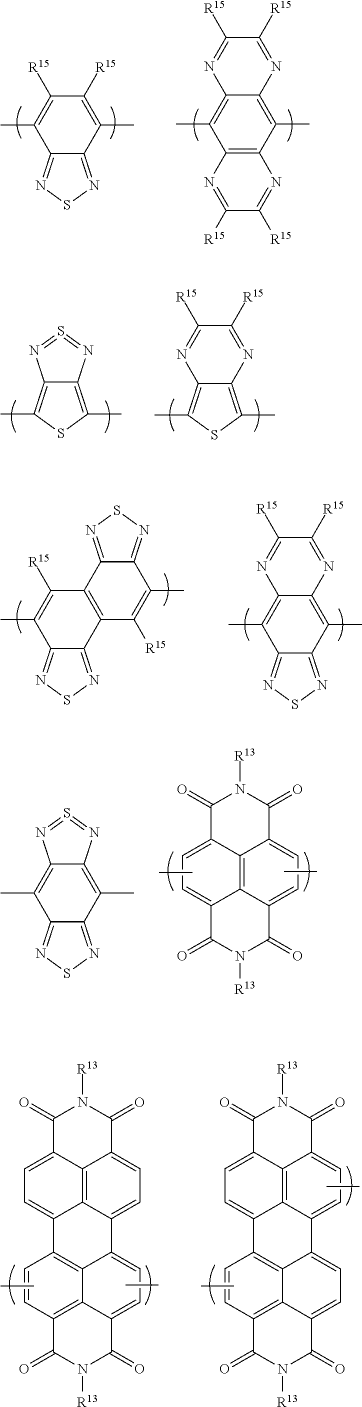

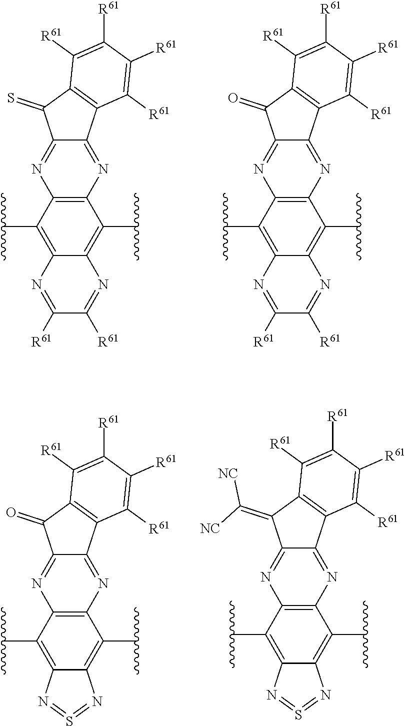

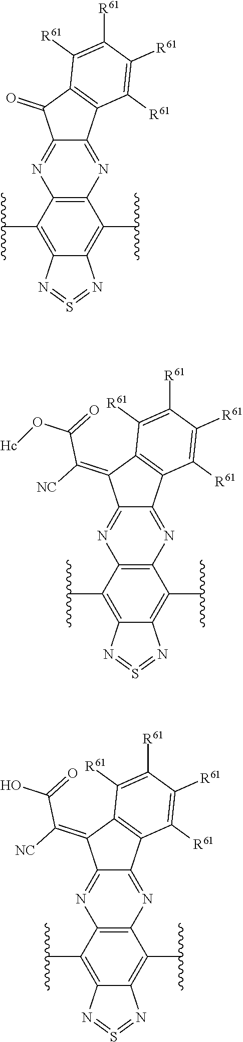

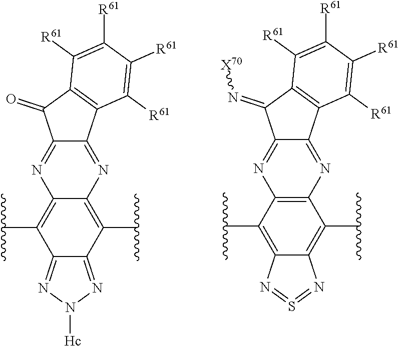

wherein Hc is a C₁₋₂₀ hydrocarbyl group, e.g., a C₁₋₂₀ alkyl group, an unsubstituted phenyl or phenyl substituted with one or more C₁₋₁₂ alkyl groups and wherein each R⁶¹ is independently H or a substituent as described above.

Exemplary donor-acceptor polymers include:

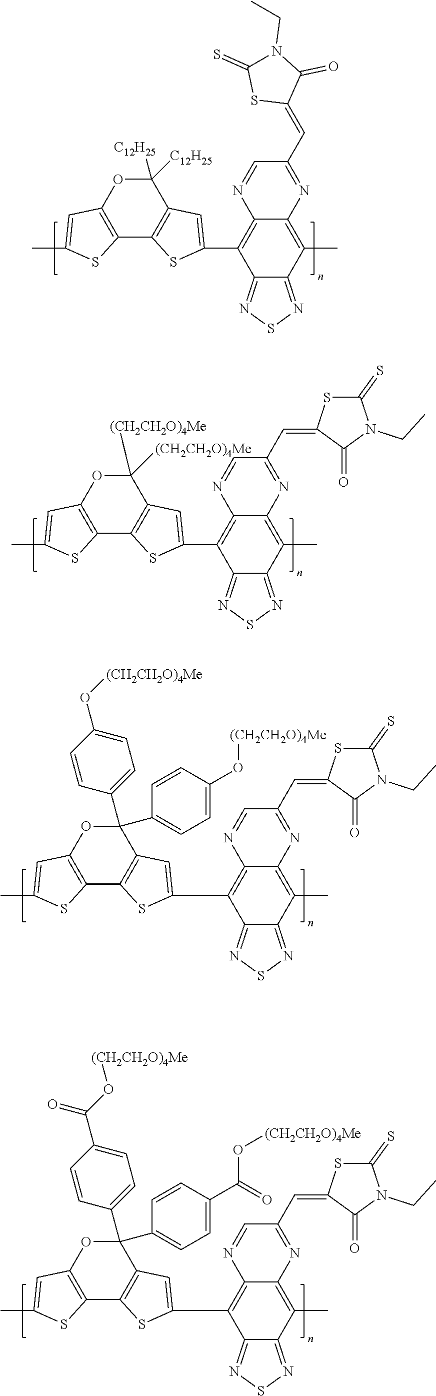

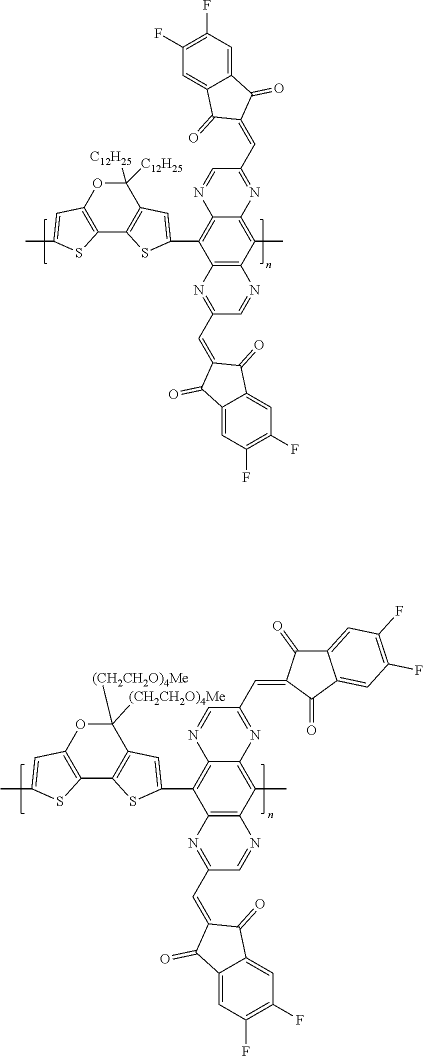

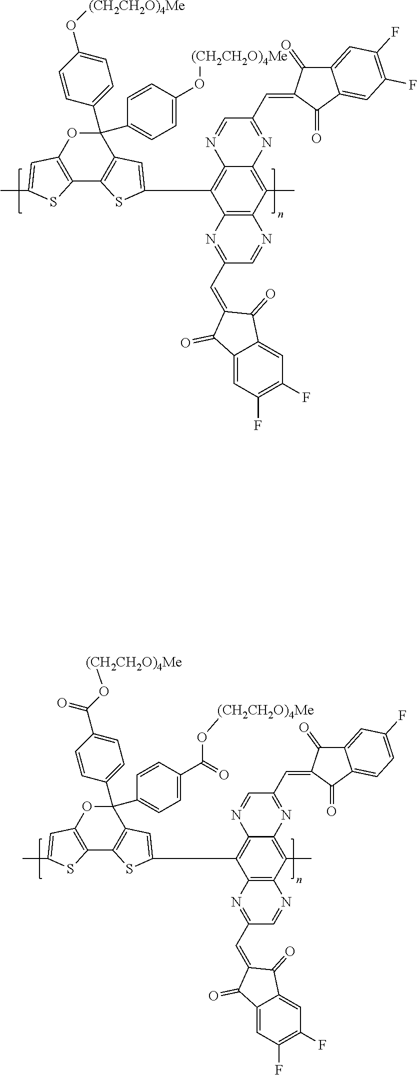

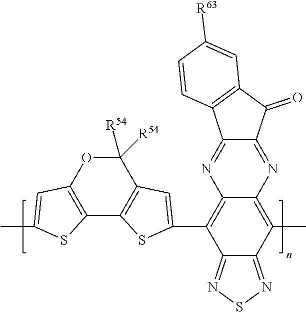

R⁵⁴ is selected from:

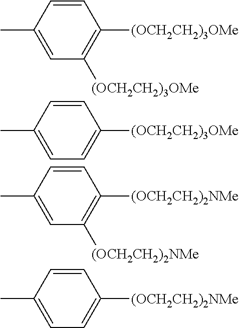

R⁶³ is selected from H, CO₂Me, —(OCH₂CH₂)₃OMe and —(OCH₂CH₂)₂NMe₂.

### Polymer Formation

The conjugated light-absorbing polymers described herein may be formed by any method known to the skilled person. Arrangement of repeat units within the polymer backbone may be controlled by, e.g., formation of block copolymers, use of polymerisation methods requiring monomers with different reactive groups; and selection of monomer ratio.

Conjugated polymers as described herein may be formed by polymerising monomers comprising leaving groups that leave upon polymerisation of the monomers to form conjugated repeat units. Exemplary polymerization methods include, without limitation, Yamamoto polymerization as described in, for example, T. Yamamoto, “Electrically Conducting And Thermally Stable pi-Conjugated Poly(arylene)s Prepared by Organometallic Processes”, Progress in Polymer Science 1993, 17, 1153-1205, the contents of which are incorporated herein by reference and Suzuki polymerization as described in, for example, WO 00/53656, WO 2003/035796, and U.S. Pat. No. 5,777,070, the contents of which are incorporated herein by reference.

The monomers may be formed by polymerisation of monomers containing boronic acid leaving groups or esters thereof, and halide or pseudo halide (e.g., sulfonate) leaving groups. The skilled person will understand that leaving groups may be selected to control which monomers may or may not form adjacent repeat units in the polymer.

### Nanoparticles

Nanoparticles comprising a light-absorbing material as described herein may be used in PAI. The nanoparticles may comprise a core comprising a light-absorbing material as described herein.

Methods of forming nanoparticles are disclosed in, for example, Kuehne et al, “Conjugated Polymer Nanoparticles toward In Vivo Theranostics—Focus on Targeting, Imaging, Therapy, and the Importance of Clearance”, Advanced Biosystems, Volume 1, Issue 11; Braeken et al, “Conjugated Polymer Nanoparticles for Bioimaging”, Materials 2017, 10, 1420; “Conjugated Polymers for Biological and Biomedical Applications”, Wiley, 25 Mar. 2018, Chapter 2, Ciftci et al, “Direct Synthesis of Conjugated Polymer Nanoparticles”, and Pecher et al, “Nanoparticles of Conjugated Polymers” Chem. Rev. 2010, 110, 10, 6260-6279, and WO 2018/060722, the contents of each of these disclosures being incorporated herein by reference.

In some embodiments, the nanoparticles are formed by precipitation from a solution of a light-absorbing polymer in a solvent. The solution may be introduced, e.g., injected, into a non-solvent which is miscible with the solvent. Optionally, the non-solvent contains a surfactant. The solution may be introduced under ultrasonication. The solvent may be evaporated to give nanoparticles dispersed in the non-solvent.

In some embodiments, the nanoparticles are formed by mini-emulsification from a solution of a light-absorbing polymer in a solvent. The solution may be mini-emulsified by addition to water or other non-solvent of the polymer in which the solvent is immiscible followed by evaporation of the solvent. The non-solvent may contain a surfactant. The mixture may be ultrasonicated.

In some embodiments, the nanoparticle is formed by self-assembly.

In some embodiments, the core comprises the light-absorbing material and a matrix. The matrix material may be an organic polymer or inorganic material. The matrix preferably comprises or consists of one or more inorganic materials, preferably silica.

In some embodiments, the nanoparticle core comprises the light-absorbing polymer and an organic polymeric matrix. The organic polymer of the matrix is preferably an amphiphilic copolymer, e.g. poly(ethyleneglycol)-methyl ether-block-poly(lactide-co-glycolide or 1,2-distearoyl-sn-glycerol-3-phosphoethanolamine-N-[methoxy(polyethyl ene glycol)-2000]. A blend of the light-absorbing material and the amphiphilic polymer may be blended to form the nanoparticle. The light-absorbing material according to these embodiments is preferably a polymer substituted with non-polar substituents.

The matrix may be mixed with (i.e., not covalently bound to) or covalently bound to the light-absorbing material. In the case of a silica matrix, the light-absorbing material may be covalently bound to the silica by a covalent linking group comprising a silane group, e.g. a group of formula —Si(OR¹¹)₃ wherein R¹¹ in each occurrence is a substituent, preferably a C₁₋₁₂ hydrocarbyl group, e.g. a C₁₋₁₂ alkyl group. An exemplary linking group is disclosed in Wang et al, “BODIPY-doped silica nanoparticles with reduced dye leakage and enhanced singlet oxygen generation”, Scientific Reports volume 5, Article number: 12602 (2015), the contents of which are incorporated herein by reference.

Covalent binding to a matrix is particularly preferred for non-polymeric light-absorbing materials which may be more prone to separation from the nanoparticle than a light-absorbing polymer.

In some embodiments, the matrix is mixed with the light-absorbing material. The light-absorbing material may be homogeneously or inhomogeneously distributed within the matrix. In the case of a light-absorbing polymer, polymer chains may be entirely contained (i.e., encapsulated) within a perimeter of the matrix material or may protrude from the perimeter.

In some embodiments, the core comprises a central region comprising the light-absorbing material and one or more shell layers surrounding the central region. The central region may consist of the light-absorbing material or may comprise one or more further materials, e.g., a matrix material as described herein mixed with or covalently bound to the light-absorbing material.

In some embodiments, the shell comprises or consists of an organic polymer, for example as disclosed in Li et al, “Polymer-encapsulated organic nanoparticles for fluorescence and photoacoustic imaging” Chem. Soc. Rev., 2014, 43, 6570-6597, the contents of which are incorporated herein by reference.

In some embodiments, the shell consists of one or more inorganic materials. The shell may consist of silica.

In some embodiments, the shell comprises an inorganic material, e.g., silica, and one or more further materials, e.g. the light-absorbing material.

The matrix and/or shell may at least partially isolate the light-absorbing material from the surrounding environment. This may limit an effect that the external environment may have on the stability or absorption properties of the light-absorbing material. In use during PAI, the matrix may limit any chemical interaction between the tissue or endogenous material and the light-absorbing material.

In some embodiments, formation of the particle core comprises polymerisation of a silica monomer, e.g. a tetraalkoxysilane, e.g. tetraethyl orthosilicate (TEOS) in the presence of the light-absorbing material. The silica monomer may be dissolved in a protic solvent, e.g., an alcohol, water or a mixture thereof. Polymerisation may be caused by addition of a base to the solution, e.g. ammonium hydroxide. Preferably according to these embodiments, the light-absorbing material is dissolved in the protic solvent. Preferably according to these embodiments, the light-absorbing material has a solubility at 25° C. and 1 atm pressure of at least 0.01 mg/ml in an alcoholic solvent, optionally, at least 0.1 or 1 mg/ml, optionally in the range of 0.01-10 mg/ml. Preferably, the alcoholic solvent is a C₁₋₁₀ alcohol, more preferably methanol. The light-absorbing material may comprise at least one of ionic and polar substituents as described herein for solubility in a protic solvent.

The surface of the nanoparticle may be functionalised with polyether groups, e.g., polyethylene glycol groups and/or binding groups as described in, for example, WO 2020/058668.

If the nanoparticle comprises a shell, a central region of the core comprising or consisting of the light-absorbing material may be formed followed by encapsulation by the shell.

Light-absorbing particles as described herein may be provided as a colloidal suspension comprising the particles suspended in a liquid. Preferably, the liquid is water, optionally a buffer solution. Preferably, the particles form a uniform (non-aggregated) colloid in the liquid.

Surface groups may be bound to a surface of the nanoparticle core. Surface groups may be selected to achieve uniform dispersion of the nanoparticles and/or to bind to a target within a tissue to be analysed, e.g. a tumour. Exemplary surface groups which may improve uniformity of dispersion and/or colloidal stability of the dispersion include, without limitation, ether-containing groups, e.g. groups containing poly(ethyleneglycol) (PEG) chains. Binding groups may be selected according to the target tissue, e.g. the binding group may be a ligand or antibody having an affinity for a cell receptor of a target cell, for example as disclosed in Mehrmohammadi et al, “Photoacoustic Imaging for Cancer Detection and Staging” Curr Mol Imaging 2013 March, 2(1): 89-105, the contents of which are incorporated herein by reference.

In some embodiments, the particles may be stored in a powder form, optionally in a lyophilised or frozen form.

Preferably, the particles have a number average diameter in the range of 1-299 nm, optionally 1-200 nm, optionally 1-50 nm, optionally 1-30 nm, optionally 1-10 nm as measured by dynamic light scattering (DLS) using a Malvern Zetasizer Nano ZS., using a 4 mW 633 nm He-Ne laser. Nanoparticle (Particle 1) suspensions in methanol can be tested in single use UV transparent plastic cuvettes. The machine was operated in Backscatter mode at an angle of 173°. Samples are equilibrated to 25° C. for 60 seconds prior to measurement. Values for the methanol solvent input into the software are 0.5476 cP for viscosity and 1.326 for the refractive index. The sample is defined as Polystyrene 10 latex (RI: 1.590, Absorption: 0.0100). The automatic measurement duration setting is used, with automatic measurement positioning and automatic attenuation. The ‘general purpose’ analysis model is used, with the default size analysis parameters along with a refractive index of 1.59 for the sample parameter. A single measurement is taken for each sample.

A size of 30 nm or below, optionally 10 nm or below is particularly advantageous in allowing excretion of the particles following introduction of the particles into a subject for PAI, in particular to allow filtration by the subject's renal system.

In some embodiments, the particles may be stored in a powder form, optionally in a lyophilised or frozen form.

### Applications

Nanoparticles as described herein may be used in PAI and in PTT.

In both PAI and PTT, nanoparticles as described herein are delivered, e.g., injected, into or onto a target region of a subject's tissue and the target region is irradiated with a laser.

In the case of PAI, energy absorbed by the light-absorbing material is converted to heat, leading to expansion and ultrasonic emission which may be detected by suitable apparatus, e.g., an ultrasonic transducer. The detected ultrasonic emission may be converted by any suitable imaging program to a 2D or 3D image of the target region. Irradiation and measurement of the photoacoustic response may be as disclosed in, for example, Zha et al, “An Ester-Substituted Semiconducting Polymer with Efficient Nonradiative Decay Enhances NIR-II Photoacoustic Performance for Monitoring of Tumor Growth”, Angewandte Chemie International Edition, 5 Sep. 2020.

Optionally, the laser has a wavelength of at least 1100 nm, optionally a wavelength in the range of 1100-1700 nm.

In the case of PTT, heat released upon irradiation may kill targeted cells, e.g. cancer cells.

PAI and PTT as described herein may be used in, for example, analysis and/or treatment of a region having a cancerous growth, e.g., tumor angiogenesis monitoring, blood oxygenation mapping, functional brain imaging, skin melanoma detection or methemoglobin measuring.

PAI and PTT as described herein may be performed on a human or non-human animal subject.

### EXAMPLES

### Modelling Data

Absorption peaks of model compounds were modelling as described in these examples was performed using Gaussian09 software available from Gaussian using Gaussian09 with B3LYP (functional). To simplify calculation, modelling was performed on compounds having methyl substituents only.

### Modelling Example 1

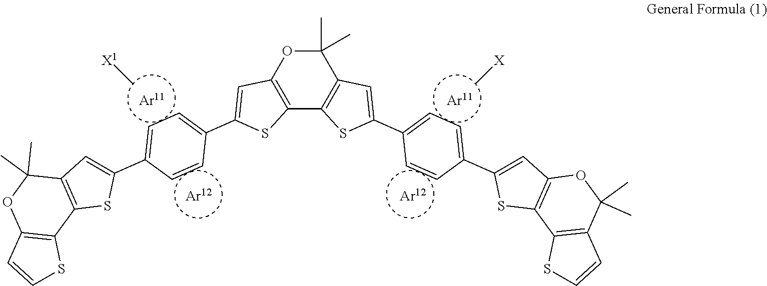

TABLE 1

| | | | | HOMO | LUMO | Eg | Abs |
|---|---|---|---|---|---|---|---|
| Entry | Ar¹¹ | Ar¹² | X¹ | (eV) | (eV) | (eV) | (nm) |
| 1-1 1-2 1-3 1-4 | 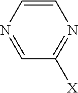 | 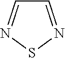 | H Rho I2F IC2F | −4.32 −4.53 −4.42 −4.58 | −3.37 −3.712 −3.75 −3.99 | 0.95 0.81 0.67 0.59 | 1304 1534 1842 2101 |
| 1-5 1-6 1-7 | 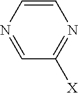 | 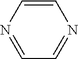 | Rho I2F IC2F | −4.77 −4.52 −4.75 | −3.74 −3.75 −4.12 | 1.03 0.78 0.63 | 1204 1600 1968 |

Rho 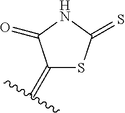 [SMILES: *=C1SC(=S)NC1=O] ICF 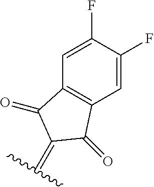 [SMILES: *=C1C(=O)c2cc(F)c(F)cc2C1=O] IC2F

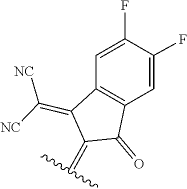 [SMILES: *=C1C(=O)c2cc(F)c(F)cc2C1=C(C#N)C#N]

### Modelling Example 2

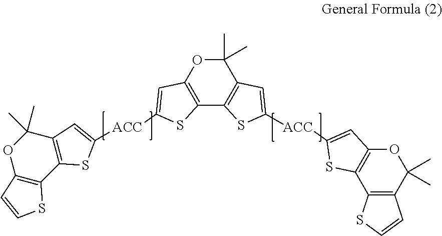

TABLE 2

| | | HOMO | LUMO | E_{g} | Abs |
|---|---|---|---|---|---|
| Entry | ACC | (eV) | (eV) | (eV) | (nm) |
| 2-1 | 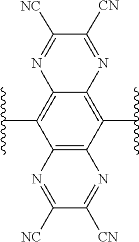 [SMILES: *c1c2nc(C#N)c(C#N)nc2c(*)c2nc(C#N)c(C#N)nc12] | −5.10 | −4.40 | 0.70 | 1779 |
| 2-2 | 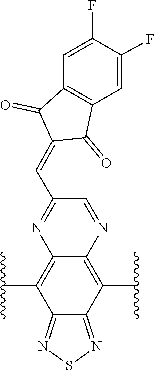 [SMILES: *c1c2ncc(C=C3C(=O)c4cc(F)c(F)cc4C3=O)nc2c(*)c2nsnc12] | −4.42 | −3.75 | 0.67 | 1842 |
| 2-3 | 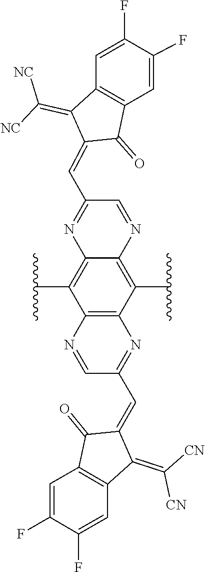 [SMILES: *c1c2ncc(/C=C3\C(=O)c4cc(F)c(F)cc4C3=C(C#N)C#N)nc2c(*)c2ncc(/C=C3\C(=O)c4cc(F)c(F)cc4C3=C(C#N)C#N)nc12] | −4.75 | −4.12 | 0.63 | 1968 |
| 2-4 | 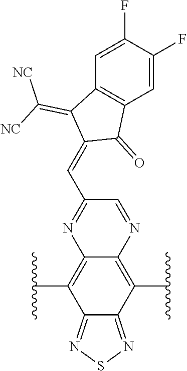 [SMILES: *c1c2ncc(/C=C3\C(=O)c4cc(F)c(F)cc4C3=C(C#N)C#N)nc2c(*)c2nsnc12] | −4.58 | −3.99 | 0.59 | 2101 |
| 2-5 | 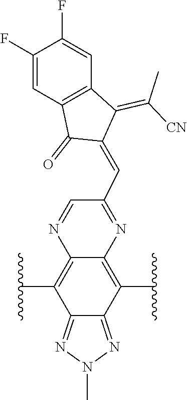 [SMILES: *c1c2ncc(/C=C3\C(=O)c4cc(F)c(F)cc4\C3=C(/C)C#N)nc2c(*)c2nn(C)nc12] | −4.46 | −3.66 | 0.80 | 1552 |

### Modelling Example 3

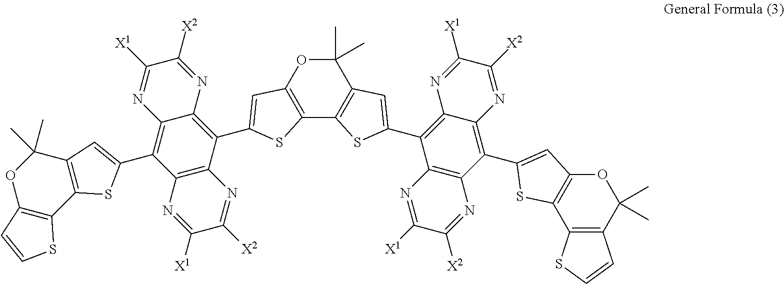

TABLE 3

| Entry | X¹ | X² | HOMO (eV) | LUMO (eV) | Eg (eV) | Abs (nm) |
|---|---|---|---|---|---|---|
| 3-1 | H | H | −4.31 | −3.16 | 1.16 | 1071 |
| 3-2 | CN | Me | −4.66 | −3.69 | 0.97 | 1274 |
| 3-3 | CO2Me | CO2Me | −4.45 | −3.48 | 0.96 | 1286 |
| 3-4 | NO2 | NO2 | −5.15 | −4.43 | 0.71 | 1739 |
| 3-5 | CN | CN | −5.10 | −4.40 | 0.70 | 1779 |
| 3-6 | 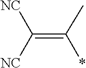 [SMILES: *C(C)=C(C#N)C#N] | 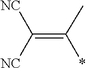 [SMILES: *C(C)=C(C#N)C#N] | −5.32 | −4.37 | 0.95 | 1306 |

### Modelling Example 4

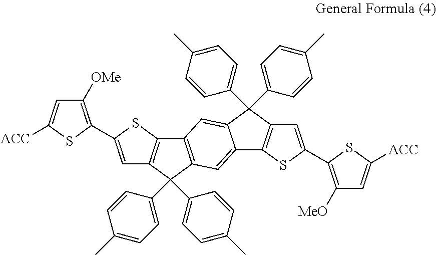

TABLE 4

| Entry | ACC | HOMO (eV) | LUMO (eV) | Eg (eV) | Abs (nm) |
|---|---|---|---|---|---|
| 4-1 | 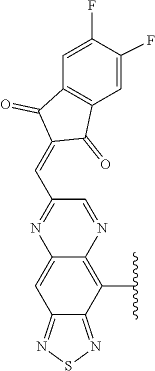 [SMILES: *c1c2ncc(C=C3C(=O)c4cc(F)c(F)cc4C3=O)nc2cc2nsnc12] | −4.58 | −3.56 | 1.02 | 1219 |
| 4-2 | 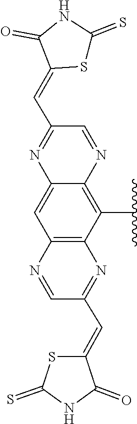 [SMILES: *c1c2ncc(/C=C3\SC(=S)NC3=O)nc2cc2ncc(/C=C3\SC(=S)NC3=O)nc12] | −4.73 | −3.74 | 0.99 | 1250 |
| 4-3 | 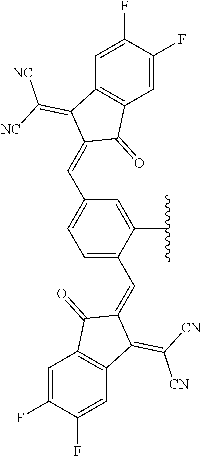 [SMILES: *c1cc(/C=C2\C(=O)c3cc(F)c(F)cc3C2=C(C#N)C#N)ccc1/C=C1\C(=O)c2cc(F)c(F)cc2C1=C(C#N)C#N] | −4.78 | −3.86 | 0.93 | 1335 |
| 4-4 | 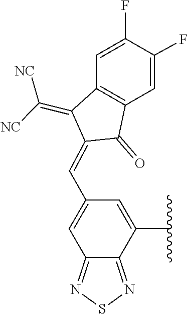 [SMILES: *c1cc(/C=C2\C(=O)c3cc(F)c(F)cc3C2=C(C#N)C#N)cc2nsnc12] | −4.57 | −3.65 | 0.92 | 1355 |
| 4-5 | 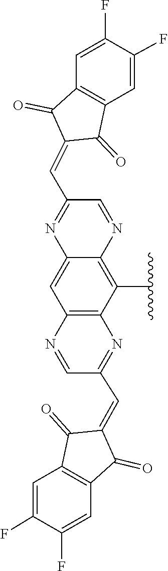 [SMILES: *c1c2ncc(C=C3C(=O)c4cc(F)c(F)cc4C3=O)nc2cc2ncc(C=C3C(=O)c4cc(F)c(F)cc4C3=O)nc12] | −4.69 | −3.78 | 0.91 | 1357 |
| 4-6 | 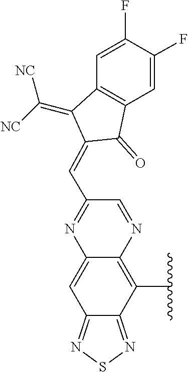 [SMILES: *c1c2ncc(/C=C3\C(=O)c4cc(F)c(F)cc4C3=C(C#N)C#N)nc2cc2nsnc12] | −4.64 | −3.79 | 0.85 | 1460 |
| 4-7 | 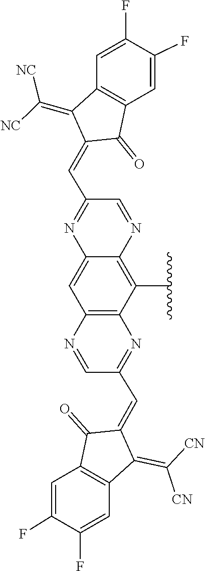 [SMILES: *c1c2ncc(/C=C3\C(=O)c4cc(F)c(F)cc4C3=C(C#N)C#N)nc2cc2ncc(/C=C3\C(=O)c4cc(F)c(F)cc4C3=C(C#N)C#N)nc12] | −4.87 | −4.03 | 0.83 | 1490 |

### Modelling Example 5

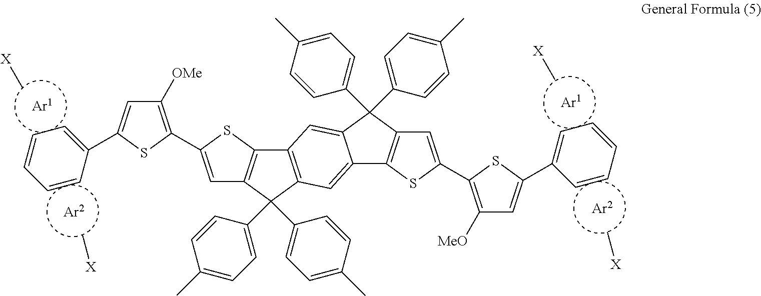

TABLE 5

| | | | HOMO | LUMO | Eg | Abs |
|---|---|---|---|---|---|---|
| Entry | Ar^{a} | X | (eV) | (eV) | (eV) | (nm) |
| 5-1 | 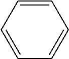 | IC2F | −4.78 | −3.86 | 0.93 | 1335 |
| 5-2 | 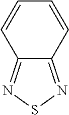 | IC2F | −4.57 | −3.65 | 0.92 | 1355 |
| 5-3 5-4 | 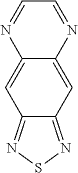 | I2F IC2F | −4.58 −4.64 | −3.56 −3.79 | 1.02 0.85 | 1219 1460 |
| 5-5 5-6 5-7 | 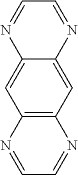 | Rho I2F IC2F | −4.73 −4.69 −4.87 | −3.74 −3.78 −4.03 | 0.99 0.91 0.83 | 1250 1357 1490 |

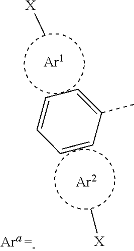

### Modelling Example 6

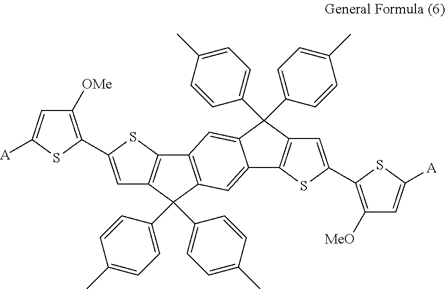

TABLE 6

| | | HOMO | LUMO | Eg | |
|---|---|---|---|---|---|
| Entry | Acceptor (A) | (eV) | (eV) | (eV) | Abs (nm) |
| 6-1 | 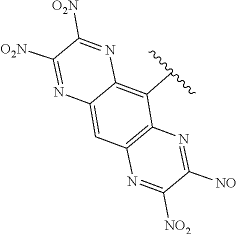 | −5.53 | −4.53 | 1.00 | 1235 |
| 6-2 | 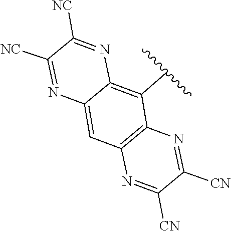 [SMILES: *c1c2nc(C#N)c(C#N)nc2cc2nc(C#N)c(C#N)nc12] | −5.38 | −4.42 | 0.97 | 1287 |

### Modelling Example 7

HOMO and LUMO levels for acceptor (ACC) of model compounds of General Formula (7) were modelled:

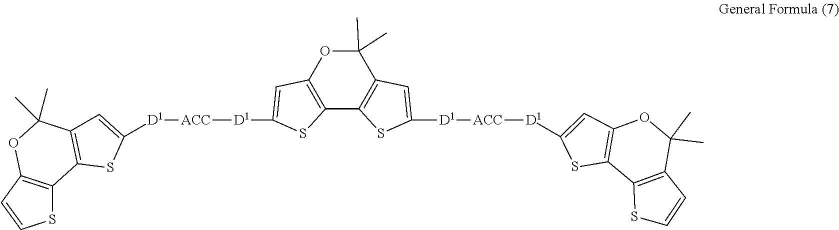

TABLE 7

| | | HOMO | LUMO | Eg | Abs |
|---|---|---|---|---|---|
| Entry | D¹-ACC-D¹ | (eV) | (eV) | (eV) | (nm) |
| Comparative Example 1-1 | 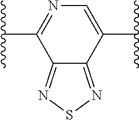 [SMILES: *c1cnc(*)c2nsnc12] | −4.60 | −3.06 | 1.54 | 803 |
| Model Example 1-1 | 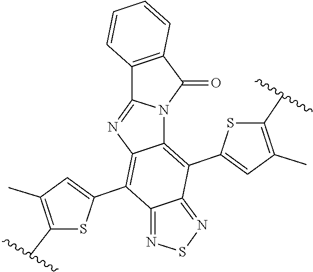 | −4.37 | −2.90 | 1.48 | 839 |
| Model Example 1-2 | 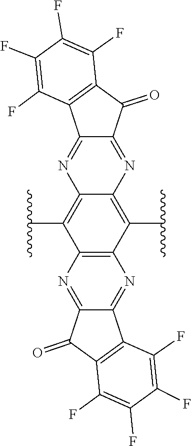 [SMILES: *c1c2nc3c(=O)c4c(F)c(F)c(F)c(F)c4c3nc2c(*)c2nc3c(=O)c4c(F)c(F)c(F)c(F)c4c3nc12] | −4.59 | −3.72 | 0.87 | 1430 |
| Model Example 1-3 | 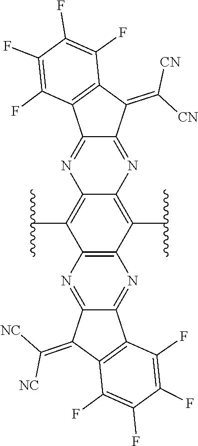 [SMILES: *c1c2nc3c(nc2c(*)c2nc4c(nc12)-c1c(F)c(F)c(F)c(F)c1C4=C(C#N)C#N)-c1c(F)c(F)c(F)c(F)c1C3=C(C#N)C#N] | −4.82 | −4.10 | 0.72 | 1724 |
| Model Example 1-4 | 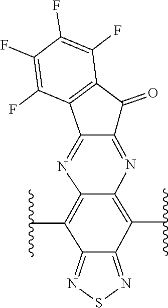 [SMILES: *c1c2nsnc2c(*)c2nc3c(nc12)C(=O)c1c(F)c(F)c(F)c(F)c1-3] | −4.54 | −3.68 | 0.87 | 1433 |
| Model Example 1-5 | 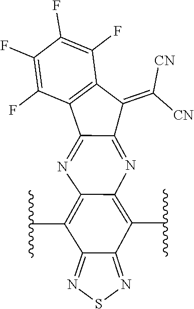 [SMILES: *c1c2nsnc2c(*)c2nc3c(nc12)C(=C(C#N)C#N)c1c(F)c(F)c(F)c(F)c1-3] | −4.67 | −3.88 | 0.79 | 1569 |
| Model Example 1-6 | 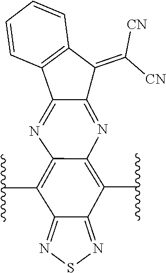 [SMILES: *c1c2nsnc2c(*)c2nc3c(nc12)C(=C(C#N)C#N)c1ccccc1-3] | −4.58 | −3.71 | 0.86 | 1437 |
| Model Example 1-7 | 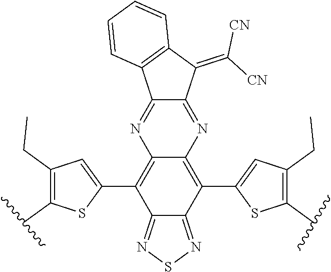 [SMILES: *c1sc(-c2c3nsnc3c(-c3cc(CC)c(*)s3)c3nc4c(nc23)C(=C(C#N)C#N)c2ccccc2-4)cc1CC] | −4.60 | −3.60 | 1.00 | 1237 |
| Model Example 1-8 | 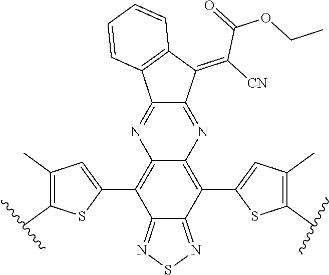 [SMILES: *c1sc(-c2c3nsnc3c(-c3cc(C)c(*)s3)c3nc4c(nc23)/C(=C(\C#N)C(=O)OCC)c2ccccc2-4)cc1C] | −4.34 | −3.43 | 0.91 | 1365 |
| Model Example 1-9 | 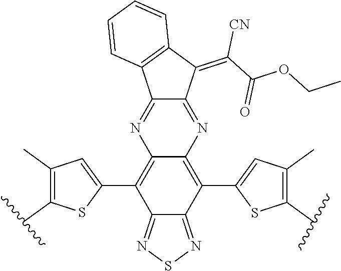 [SMILES: *c1sc(-c2c3nsnc3c(-c3cc(C)c(*)s3)c3nc4c(nc23)/C(=C(/C#N)C(=O)OCC)c2ccccc2-4)cc1C] | −4.47 | −3.48 | 0.99 | 1255 |
| Model Example 1-10 | 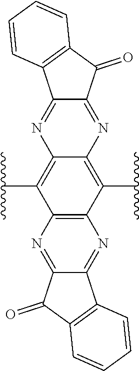 [SMILES: *c1c2nc3c(=O)c4ccccc4c3nc2c(*)c2nc3c(=O)c4ccccc4c3nc12] | −4.44 | −3.40 | 1.04 | 1190 |
| Model Example 1-11 | 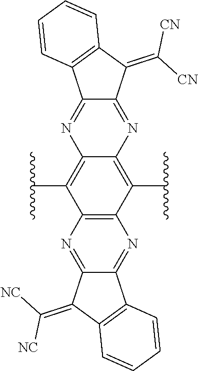 [SMILES: *c1c2nc3c(nc2c(*)c2nc4c(nc12)-c1ccccc1C4=C(C#N)C#N)-c1ccccc1C3=C(C#N)C#N] | −4.65 | −3.88 | 0.77 | 1608 |
| Model Example 1-12 | 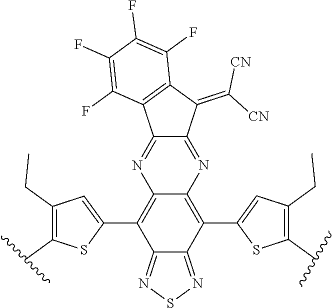 [SMILES: *c1sc(-c2c3nsnc3c(-c3cc(CC)c(*)s3)c3nc4c(nc23)C(=C(C#N)C#N)c2c(F)c(F)c(F)c(F)c2-4)cc1CC] | −4.64 | −3.77 | 0.87 | 1419 |
| Model Example 1-13 | 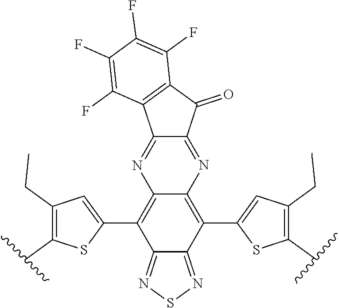 [SMILES: *c1sc(-c2c3nsnc3c(-c3cc(CC)c(*)s3)c3nc4c(nc23)C(=O)c2c(F)c(F)c(F)c(F)c2-4)cc1CC] | −4.53 | −3.53 | 1.00 | 1242 |
| Model Example 1-14 | 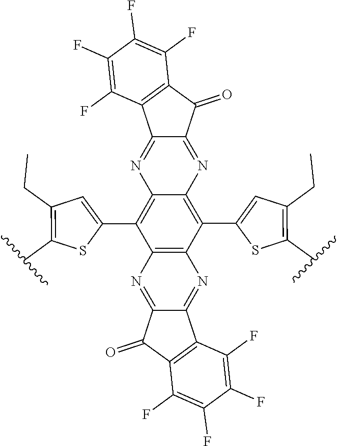 [SMILES: *c1sc(-c2c3nc4c(=O)c5c(F)c(F)c(F)c(F)c5c4nc3c(-c3cc(CC)c(*)s3)c3nc4c(=O)c5c(F)c(F)c(F)c(F)c5c4nc23)cc1CC] | −4.51 | −3.58 | 0.93 | 1340 |
| Model Example 1-15 | 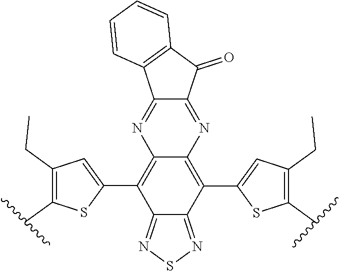 [SMILES: *c1sc(-c2c3nsnc3c(-c3cc(CC)c(*)s3)c3nc4c(nc23)C(=O)c2ccccc2-4)cc1CC] | −4.45 | −3.35 | 1.10 | 1128 |
| Model Example 1-16 | 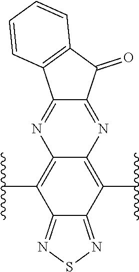 [SMILES: *c1c2nsnc2c(*)c2nc3c(nc12)C(=O)c1ccccc1-3] | −4.43 | −3.48 | 0.95 | 1306 |
| Model Example 1-17 | 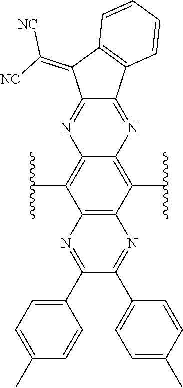 [SMILES: *c1c2nc3c(nc2c(*)c2nc(-c4ccc(C)cc4)c(-c4ccc(C)cc4)nc12)-c1ccccc1C3=C(C#N)C#N] | −4.47 | −3.43 | 1.04 | 1194 |
| Model Example 1-18 | 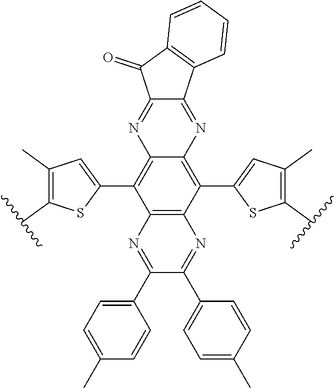 [SMILES: *c1sc(-c2c3nc4c(nc3c(-c3cc(C)c(*)s3)c3nc(-c5ccc(C)cc5)c(-c5ccc(C)cc5)nc23)-c2ccccc2C4=O)cc1C] | −4.28 | −3.10 | 1.18 | 1051 |
| Model Example 1-19 | 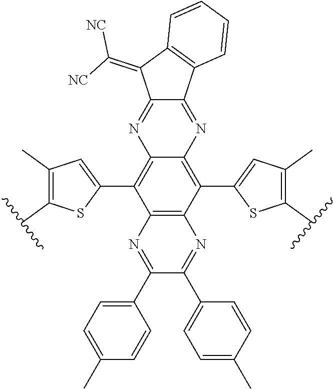 [SMILES: *c1sc(-c2c3nc4c(nc3c(-c3cc(C)c(*)s3)c3nc(-c5ccc(C)cc5)c(-c5ccc(C)cc5)nc23)-c2ccccc2C4=C(C#N)C#N)cc1C] | −4.41 | −3.38 | 1.03 | 1201 |
| Model Example 1-20 | 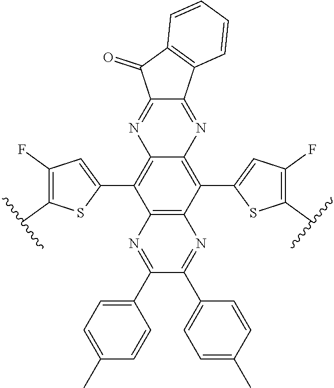 [SMILES: *c1sc(-c2c3nc4c(nc3c(-c3cc(F)c(*)s3)c3nc(-c5ccc(C)cc5)c(-c5ccc(C)cc5)nc23)-c2ccccc2C4=O)cc1F] | −4.39 | −3.27 | 1.12 | 1107 |
| Model Example 1-21 | 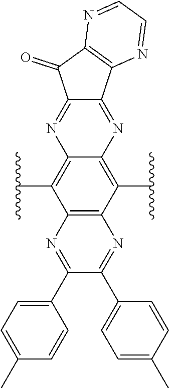 [SMILES: *c1c2nc3c(nc2c(*)c2nc(-c4ccc(C)cc4)c(-c4ccc(C)cc4)nc12)-c1nccnc1C3=O] | −4.39 | −3.33 | 1.06 | 1172 |
| Model Example 1-22 | 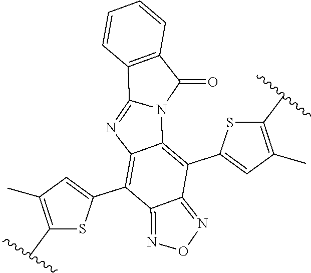 | −4.52 | −2.92 | 1.60 | 773 |
| Model Example 1-23 | 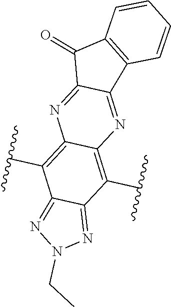 [SMILES: *c1c2nc3c(nc2c(*)c2nn(CC)nc12)-c1ccccc1C3=O] | −4.34 | −3.06 | 1.28 | 966 |
| Model Example 1-24 | 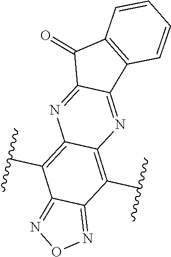 [SMILES: *c1c2nonc2c(*)c2nc3c(nc12)C(=O)c1ccccc1-3] | −4.54 | −3.60 | 0.93 | 1320 |
| Model Example 1-25 | 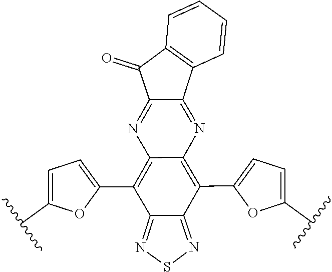 [SMILES: *c1ccc(-c2c3nsnc3c(-c3ccc(*)o3)c3nc4c(nc23)C(=O)c2ccccc2-4)o1] | −4.35 | −3.30 | 1.05 | 1181 |
| Model Example 1-26 | 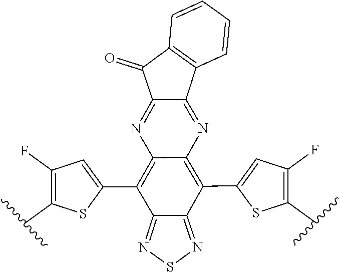 [SMILES: *c1sc(-c2c3nsnc3c(-c3cc(F)c(*)s3)c3nc4c(nc23)C(=O)c2ccccc2-4)cc1F] | −4.45 | −3.56 | 0.90 | 1384 |
| Model Example 1-27 | 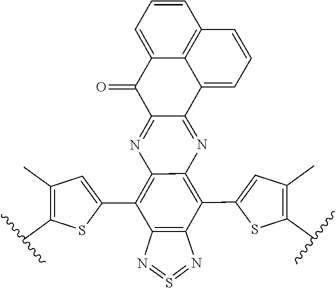 [SMILES: *c1sc(-c2c3c(c(-c4cc(C)c(*)s4)c4nc5c(nc24)C(=O)c2cccc4cccc-5c24)N=S=N3)cc1C] | −4.34 | −3.39 | 0.95 | 1311 |
| Model Example 1-28 | 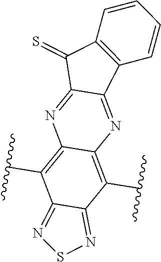 [SMILES: *c1c2nsnc2c(*)c2nc3c(nc12)C(=S)c1ccccc1-3] | −3.59 | −4.43 | 0.84 | 1480 |
| Model Example 1-29 | 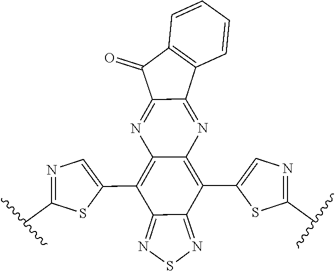 [SMILES: *c1ncc(-c2c3nsnc3c(-c3cnc(*)s3)c3nc4c(nc23)C(=O)c2ccccc2-4)s1] | −4.67 | −3.53 | 1.134 | 1091 |
| Model Example 1-30 | 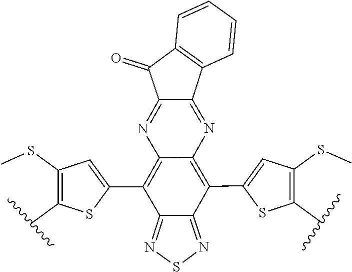 | −4.70 | −3.49 | 1.21 | 1025 |
| Model Example 1-31 | 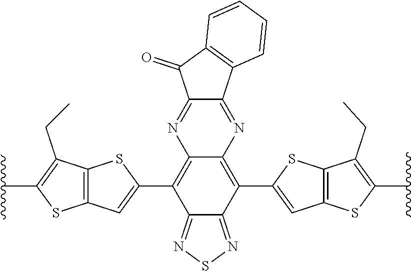 [SMILES: *c1sc2cc(-c3c4nsnc4c(-c4cc5sc(*)c(CC)c5s4)c4nc5c(nc34)C(=O)c3ccccc3-5)sc2c1CC] | −4.60 | −3.43 | 1.18 | 1053 |
| Model Example 1-32 | 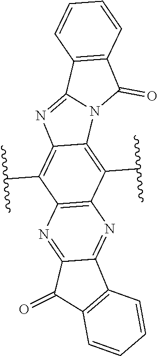 [SMILES: *c1c2nc3c(=O)c4ccccc4c3nc2c(*)c2c1nc1c3ccccc3c(=O)n12] | −4.48 | −2.87 | 1.61 | 770 |
| Model Example 1-33 | 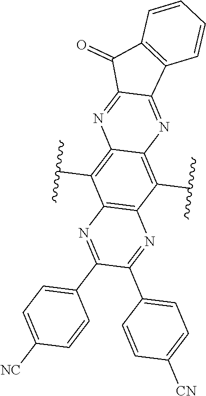 [SMILES: *c1c2nc3c(nc2c(*)c2nc(-c4ccc(C#N)cc4)c(-c4ccc(C#N)cc4)nc12)-c1ccccc1C3=O] | −4.65 | −3.50 | 1.16 | 1073 |
| Model Example 1-34 | 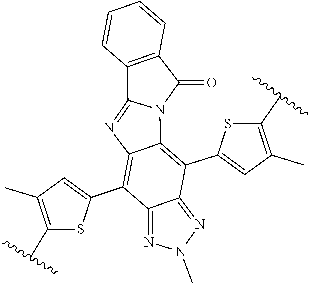 | −4.40 | −2.53 | 1.87 | 662 |
| Model Example 1-35 | 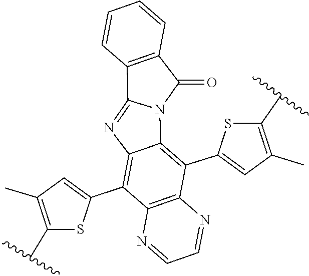 | −4.37 | −2.75 | 1.62 | 767 |
| Model Example 1-36 | 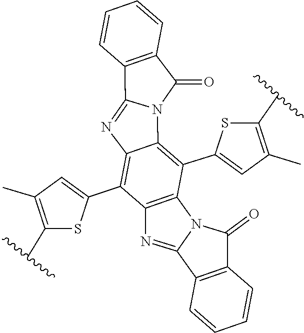 | −4.50 | −2.43 | 2.06 | 601 |
| Model Example 1-37 | 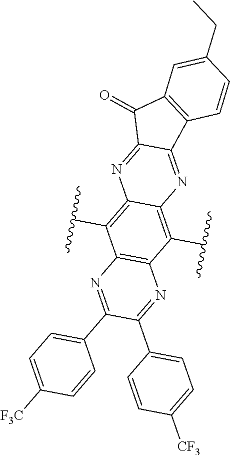 [SMILES: *c1c2nc3c(nc2c(*)c2nc(-c4ccc(C(F)(F)F)cc4)c(-c4ccc(C(F)(F)F)cc4)nc12)-c1ccc(CC)cc1C3=O] | −4.49 | −3.30 | 1.19 | 1045 |
| Model Example 1-38 | 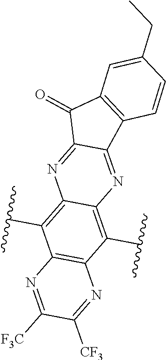 [SMILES: *c1c2nc3c(nc2c(*)c2nc(C(F)(F)F)c(C(F)(F)F)nc12)-c1ccc(CC)cc1C3=O] | −4.55 | −3.55 | 1.00 | 1237 |
| Model Example 1-39 | 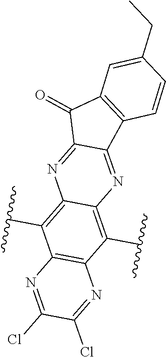 [SMILES: *c1c2nc(Cl)c(Cl)nc2c(*)c2nc3c(nc12)C(=O)c1cc(CC)ccc1-3] | −4.51 | −3.40 | 1.11 | 1119 |
| Model Example 1-40 | 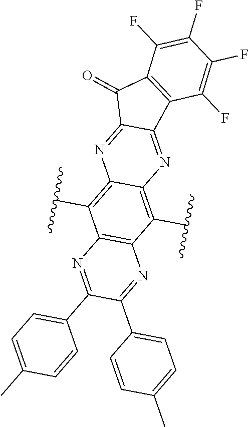 [SMILES: *c1c2nc3c(nc2c(*)c2nc(-c4ccc(C)cc4)c(-c4ccc(C)cc4)nc12)-c1c(F)c(F)c(F)c(F)c1C3=O] | −4.42 | −3.29 | 1.12 | 1104 |
| Model Example 1-41 | 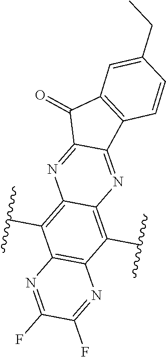 [SMILES: *c1c2nc(F)c(F)nc2c(*)c2nc3c(nc12)C(=O)c1cc(CC)ccc1-3] | −4.50 | −3.29 | 1.20 | 1029 |
| Model Example 1-42 | 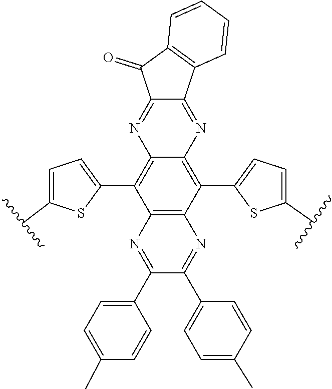 [SMILES: *c1ccc(-c2c3nc4c(nc3c(-c3ccc(*)s3)c3nc(-c5ccc(C)cc5)c(-c5ccc(C)cc5)nc23)-c2ccccc2C4=O)s1] | −4.36 | −3.10 | 1.26 | 985 |

### Intermediate Compound 1

Intermediate Compound 1 may be formed by the method as described in WO 2011/052709 in which lactone intermediate 4 is reacted with the following Grignard reagent:

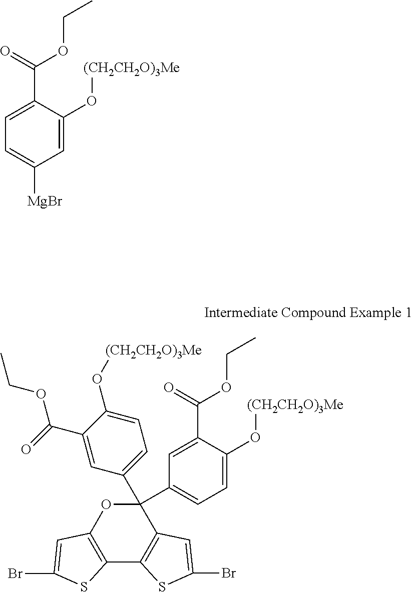

### Intermediate Compound Example 2

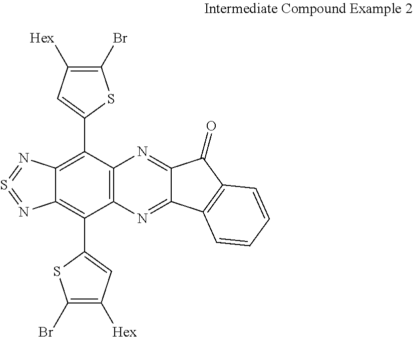

Intermediate Compound Example 2 was prepared following the reported procedure in Macromolecules 2019, 52, p 6149-6159.

### Intermediate Compound Example 3

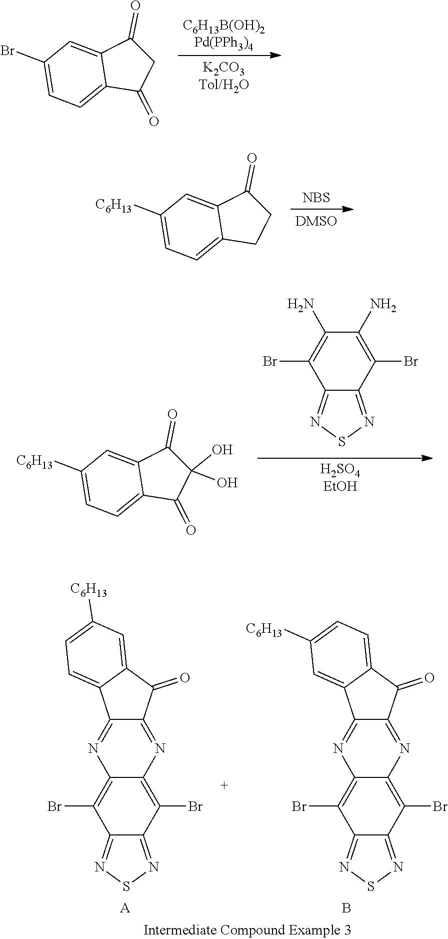

A solution of K₂CO₃ (23.31 g, 169 mmol) in water (70 ml) was added to a degassed solution of 6-bromoindanone (7.12 g, 33.73 mmol), hexyl-boronic acid (8.77 g, 67.47 mmol), Pd(PPh₃)₄ (3.40 g, 3.37 mmol) and toluene. After refluxing overnight, the reaction mixture was cooed to room temperature, separated and the aqueous phase washed with toluene (50 ml). The combined organic layers were dried, evaporated and purified via column chromatography (heptane/ethyl acetate) to give 6-hexyll-indanone (3.38 g) as an oil.

A solution of 6-hexyl-1-indanone (3.38 g, 15.62 mmol) and N-bromosuccinimide (5.7 g, 32.03 mmol) in DMSO was heated at 80° C. for 6 hrs. The cooled reaction mixture was poured into water (200 ml) and extracted with ethyl acetate, dried, evaporated and purified via recrystallization from heptane/ethyl acetate to give 6-hexyl ninhydrin (1.34 g).

Concentrated sulfuric acid (5 drops) was added to a solution of 6-hexyl-ninhydrin (1.0 g. 3.09 mmol) and 4,7-dibromo-5,6-diamino-2,1,3-benzothiadiazole (1.21 g, 4.63 mmol, prepared as described in Bioconjugate Chemistry, 2016, 27(7), p 1614-1623) in ethanol (35 ml). After heating at 80° C. overnight, the reaction mixture was cooled to room temperature, the solid filtered and washed with water, ethanol, methanol and recrystallized (chloroform/methanol) to give Intermediate Compound Example 3 (1.13 g) as a mixture of 2 isomers.
Isomer A: ¹H NMR (400 MHz, CDCl₃), δ [ppm]: 8.27 (d, 1H, 7.0 Hz); 7.89 (s, 1H); 7.72 (d, 1H, 7.2 Hz); 2.81 (t, 2H, 7.8 Hz), 1.71-1.78 (m, 2H); 1.3-1.4 (m, 6H); 1.72 (m, 3H)
Isomer B: ¹H NMR (400 MHz, CDCl₃), δ [ppm]: 8.17 (s, 1H); 7.99 (d, 1H, 7.8 Hz); 7.60 (d, 1H, 7.9 Hz); 2.86 (t, 2H, 8.0 Hz); 1.71-1.78 (m, 2H), 1.3-1.4 (m, 6H); 1.72 (m, 3H)

### Intermediate Compound Example 4

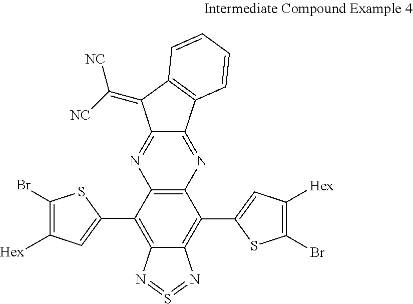

Intermediate Compound Example 4 was prepared following the reported procedure in Macromolecules 2019, 52, p 6149-6159.

Intermediate Compound Example 5

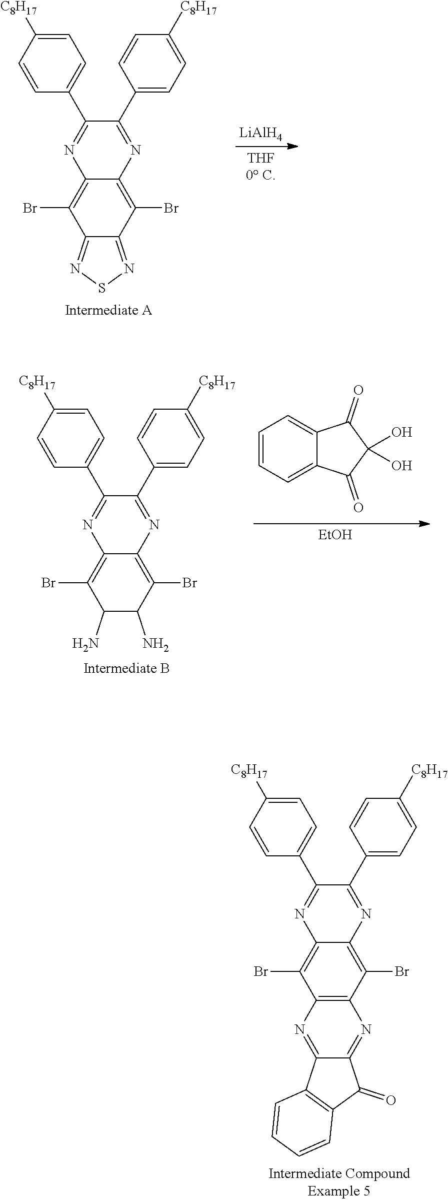

A solution of Intermediate A (6.85 g, 9.48 mmol, prepared as described in Bioconjugate Chemistry, 2016, 27(7), p 1614-1623) in THF (257 ml) of THF was cooled to 0° C. LiAlH₄ (37.92 ml, 37.92 mmol, 1M in THF) was added dropwise. After 30 minutes the reaction mixture was quenched with water, evaporated dissolved in ethyl acetate and filtered. Precipitation from heptane gave Intermediate B (4.24 g) as a yellow solid.

A solution of Intermediate B (4.24 g, 5.87 mmol) and ninhydrin (4.18 g, 23.47 mmol) in ethanol (20 ml) was heated at reflux overnight. The reaction mixture was cooled, and the resulting orange precipitate was filtered, washed with ethanol and recrystallized (isopropanol/chloroform) to give Intermediate Compound Example 5 (3.70 g).

¹H NMR (400 MHz, CDCl₃), δ [ppm]: 8.38 (d, 1H, 7.6Hz); 8.05 (d, 1H, 7.8 Hz); 7.88 (t, 1H, 7.5 Hz); 7.69-7.74 (m, 5H); 7.20-7.22 (m, 4H); 2.64-2.67 (m, 4H); 1.61-1.65 (m, 4H); 1.27-1.32 (m, 20H); 0.88 (t, 6H, 7.1Hz).

### Intermediate Compound Example 6

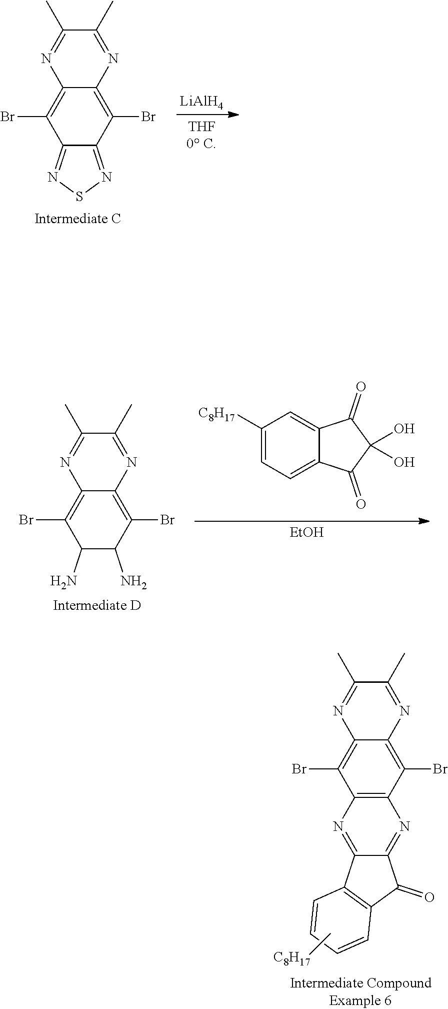

Intermediate C (4.80 g; 12.83, 1 eq.) was dissolved in 120 ml of dry THF under nitrogen atmosphere. This solution was cooled to −20° C. and LiAlH₄ (1M in THF; 12.83 mL) was added dropwise over 10 minutes. The reaction mixture was slowly brought to room temperature and stirred for 3.5 hours, cooled to 0° C. and quenched with 5 mL of water. Solvent was removed and the residue was extracted with ethyl acetate, filtered, diluted with heptane and evaporated. The product was recrystallized from ethyl acetate/methanol mixture to give 2.63 g of intermediate D.

Ninhydrin-C8 (2.77 g; 9.28 mmol) dissolved in warm ethanol (75 ml) and Intermediate D was added, and the mixture was heated to reflux overnight. The mixture was brought to room temperature, filtered, and washed with methanol and heptane. The product was purified via column chromatography (reverse phase-C18; MeCN/THF; 4%-36%), and filtered from methanol to give intermediate compound 6 0.54 gas a mixture of 2 isomers (ratio 1:3).

¹H NMR (400 MHz, CDCl₃), δ [ppm]: 8.23 (d, 1H); 8.14 (s, 1H); 7.94 (d, 1H); 7.85 (s, 1H); 7.67 (d, 1H); 7.53 (d, 1H); 2.92 (s, 6H); 2.85 (t, 2H); 2.78 (t, 2H); 1.70-1.78 (m, 2H); 1.29-1.56 (m, 10H), 0.88 (t, 3H).

### Intermediate Compound Example 7

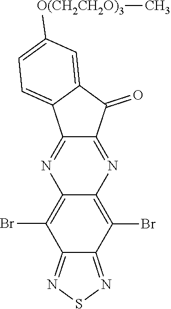

### Intermediate Compound Examples 8 and 9

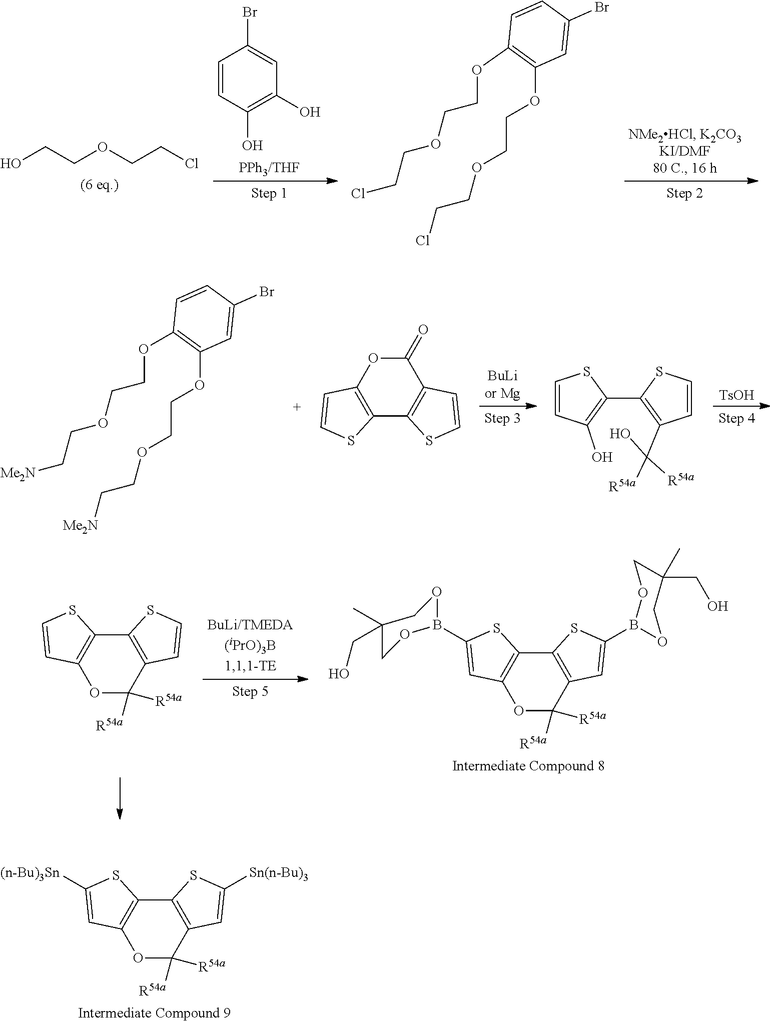

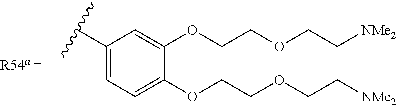 [SMILES: *c1ccc(OCCOCCN(C)C)c(OCCOCCN(C)C)c1]

In Step 1, 6 equivalents of 2-(2-chloroethoxy)ethanol (6 equivalents) in THF is cooled to 0° C., 6 equivalents of diisopropyl azodicarboxylate is added dropwise and warmed up overnight, the solvent is removed and the product is purified by column chromatography with ethyl acetate/petrol ether eluant.

In Step 5, following reaction the reaction mixture was acidified and the aqueous layer was removed. 1,1,1-Trimethylolethane was added to form the diester.

Intermediate Compound Examples 1-9 may be reacted to form polymeric or non-polymeric materials comprising an electron-accepting unit derived from these compounds and an electron-donating unit.

Polymers may be formed by Suzuki-Miyaura polymerisation of Intermediate Compound Examples 1-6 with a monomer for forming an electron-donating repeat unit, for example as disclosed in US9512149, the contents of which are incorporated herein by reference.

### Polymer Examples 1 and 2

Polymer Examples may be synthesized as described in WO2011052709 and WO2013051676, the contents of which are incorporated herein by reference, by polymerisation of Monomer Example 1 and a monomer for forming an electron-accepting repeat unit. In the case of Polymer Examples 1 and 2, polymerisation is followed by hydrolysis of the ester group of Monomer Example 1 to —COO⁻Cs⁺, as described in WO 2012/133229, the contents of which are incorporated herein by reference.

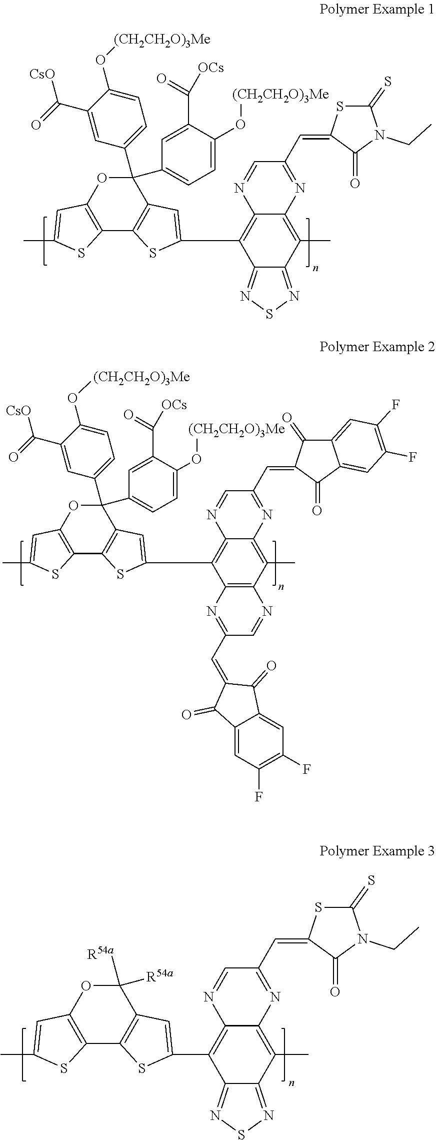

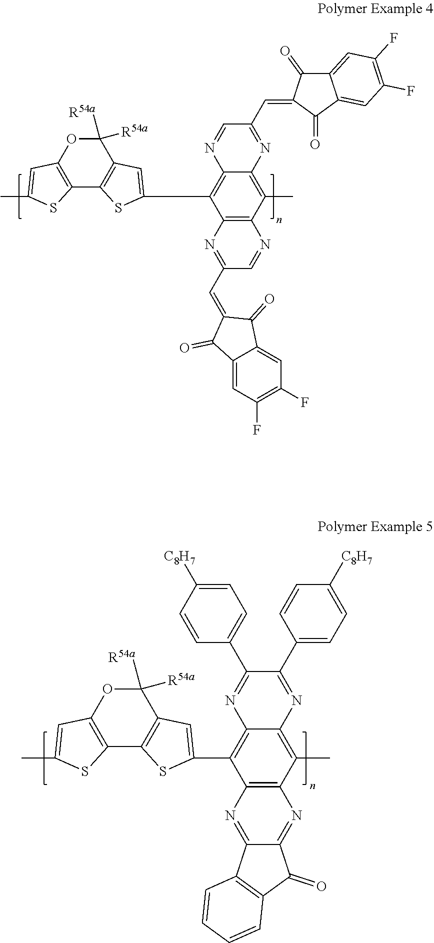

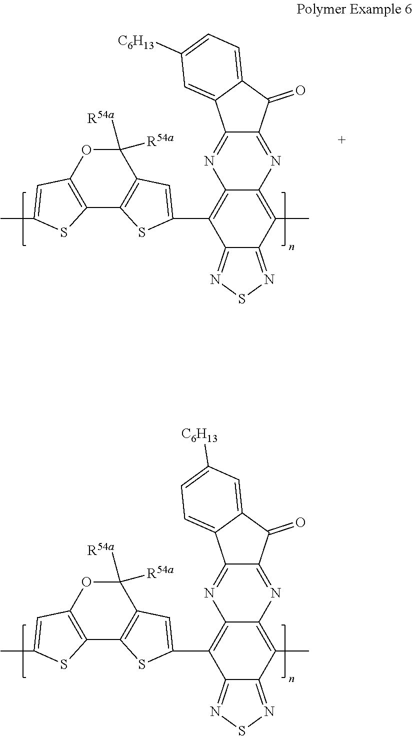

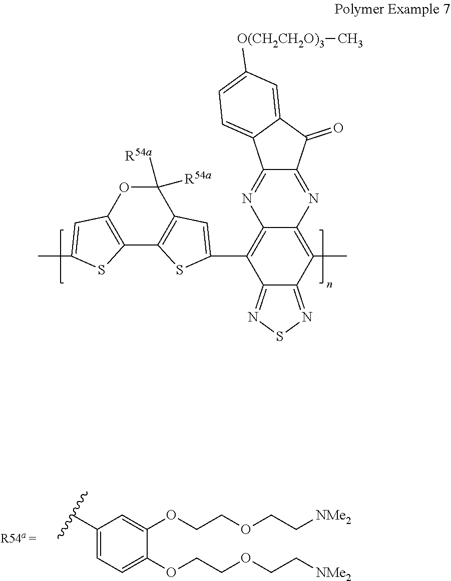

### Polymer Band Gap and Absorption

Polymer HOMO levels, LUMO levels and absorption maxima were measured for donor-acceptor polymers containing alternating donor and acceptor repeat units wherein the acceptor repeat units are as shown in Table 8 and the donor repeat units have formula:

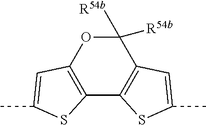

in which R^{54b} of 50 mol % of the donor repeat units is 3,7-dimethyloctyl and R^{54b} for 50 mol % of the donor repeat units is C₁₂H₂₅.

HOMO and LUMO levels were measured in solution square wave voltammetry.

In square wave voltammetry, the current at a working electrode is measured while the potential between the working electrode and a reference electrode is swept linearly in time. The difference current between a forward and reverse pulse is plotted as a function of potential to yield a voltammogram. Measurement may be with a CHI 660D Potentiostat.

The apparatus to measure HOMO or LUMO energy levels by SWV may comprise a cell containing 0.1 M tertiary butyl ammonium hexafluorophosphate in acetonitrile; a 3 mm diameter glassy carbon working electrode; a platinum counter electrode and a leak free Ag/AgCl reference electrode.

Ferrocene is added directly to the existing cell at the end of the experiment for calculation purposes where the potentials are determined for the oxidation and reduction of ferrocene versus Ag/AgCl using cyclic voltammetry (CV).

LUMO=4.8-E ferrocene(peak to peak average)−E reduction of sample(peak maximum).

HOMO=4.8-E ferrocene(peak to peak average)+E oxidation of sample(peak maximum).

The HOMO and LUMO values were measured from a dilute solution of the polymers (0.3 wt. %) in toluene.

### Absorption Spectra were Measured in Toluene Solution

TABLE 8

| | | | | | Abs max/ | Abs max/ |
|---|---|---|---|---|---|---|
| | | HOMO/ | LUMO/ | Band | nm | nm |
| Polymer | Acceptor unit | eV | eV | gap | (solution) | (film) |
| 8 | 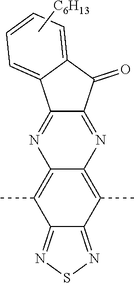 | −4.91 | −3.92 | 0.99 | 1663 | 1535 |
| 9 | 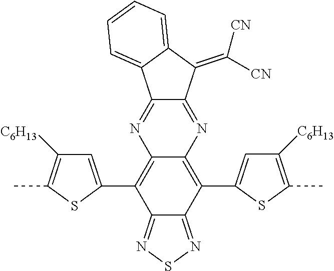 | −5.1 | −3.95 | 1.15 | 1390 | 1266 |
| 10 | 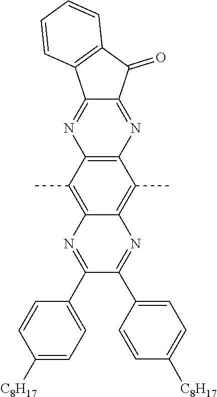 | −4.84 | −3.74 | 1.10 | 1297 | 1230 |
| 11 | 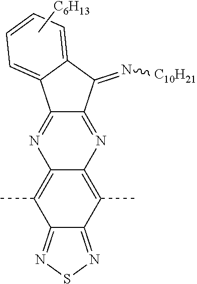 | −4.88 | −3.69 | 1.19 | 1404 | 1297 |
| 12 | 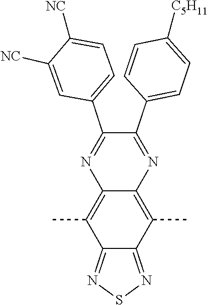 | −4.86 | −3.93 | 0.93 | 1582 | 1429 |
| 13 | 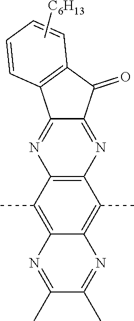 | −4.88 | −3.63 | 1.25 | 1189 | 1136 |
| 14 | 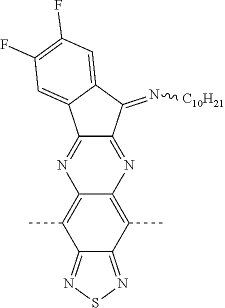 | −4.81 | −3.55 | 1.26 | 1573 | 1489 |
| 15 | 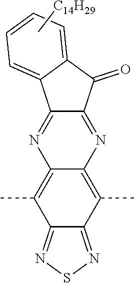 | −5.00 | −3.80 | 1.2 | 1722 | 1660 |
| 16 | 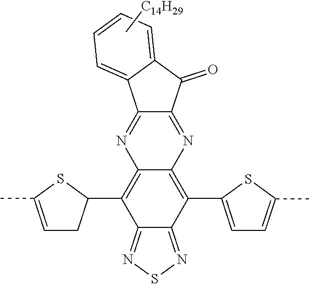 | −5.06 | −3.75 | 1.31 | 1235 | 1238 |
| 17 | 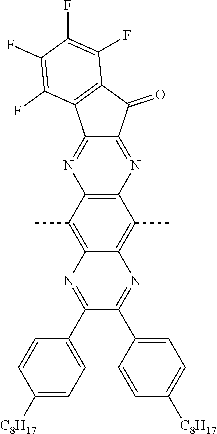 | −4.94 | −3.84 | 1.1 | 1484 | 1341 |

### Nanoparticle Formation—Example 1

The nanoparticle can be synthesised as described in GB2554666, the contents of which are incorporated herein by reference. A polymer (e.g. Polymer Example 1 or 2) is dissolved in methanol (either 1 mg/mL or 10 mg mL) by heating to 60° C. for 1 hour and the solution is then cooled to room temperature. To 2 mL of this solution is added (ammonium hydroxide (0.15 mL, 30% aq.), followed by rapid addition of a solution comprised of tetraethylorthosilicate (TEOS, 0.2 mL) and methanol (0.5 mL), with stirring at room temperature. Stirring is continued for 1 h at room temperature, after which time the solution is centrifuged at 14,000 rpm for 10 minutes to isolate the resultant silica-polymer nanoparticles from the supernatant containing excess unreacted TEOS and ammonium hydroxide. The supernatant is decanted and gentle sonication is used to redisperse the isolated pellet of nanoparticles in methanol (2.5 mL). Wash cycles consisting of centrifugation, decanting and redispersion in methanol (2.5 mL) are repeated a further two times, followed by three similar washes with deionised water (2.5 mL). Finally, the nanoparticles are redispersed in deionised water (1.5 mL) for measurement of particle size via dynamic light scattering using a Malvern Zetasizer Nano ZS.

### Nanoparticle Formation—Example 2

Nanoparticles were formed following process from Colloids and Surfaces B: Biointerfaces, 2012, 91, 219-225, the content of which is incorporated herein by reference.

40 mL amber vials were charged with cyclohexane (3.75 mL), hexanol (1.25 mL), Triton X-100 (0.85 mL) and MILLI-Q water (0.25 mL). This was stirred for 5-10 mins at 500 rpm with a 12×4.5 mm stirrer bar. Then TEOS or TMOS (50 microlitres), polymer solution (25 or 50 microlitres of a solution of Polymer 8 illustrated in Table 8 in toluene at 1 mg/mL) and ammonium hydroxide (28-30%, 30 microlitres) were added. The vials were sealed and stirred at room temperature for about 20 hours.

After 20 hours, aliquots from each batch were diluted into methanol (200 μL crude into 800 μL methanol) and analysed by DLS. The DLS samples were poured back into the reaction vials along with a further 5 mL methanol and stirred for about 6 hours to break the micelles. The samples were then loaded into pre-conditioned Float-a-lyzer dialysis devices (10 mL, 100 kDa MWCO). Batches 2 and 4 were passed through 0.45 μm filters during the transfer (batches 1 and 3 would not pass through 0.45 μm filters) and dialysed against ethanol for 2 hours. The dialysis solvent was then changed to water and the samples were dialysed overnight (black precipitate that was visible after ethanol dialysis became homogeneous suspension again overnight). The following day the dialysis water was changed 3 further times (every 2-3 hours). The samples were measured by DLS.

| | | Polymer | | | | | |
|---|---|---|---|---|---|---|---|
| | | 8 | | Z | N | | I |
| | Sili- | quantity | | ave/ | ave/ | | ave/ |
| Batch | cate | (μL) | Stage | nm | nm | PDI | nm |
| 1 | TEOS | 25 | Crude | 206.0 | 154.9 | 0.100 | 232.3 |
| | | | Dialysed | 182.5 | 162.7 | 0.171 | 213.4 |
| 2 | TMOS | 25 | Crude | 36.31 | 24.02 | 0.267 | 36.77* |
| | | | Dialysed | 48.89 | 8.73 | 0.506 | 93.39* |
| 3 | TEOS | 50 | Crude | 208.0 | 145.5 | 0.168 | 233.7 |
| | | | Dialysed | 180.9 | 127.3 | 0.125 | 207.4 |
| 4 | TMOS | 50 | Crude | 82.88 | 24.96 | 0.215 | 32.01* |
| | | | Dialysed | 89.24 | 9.085 | 0.567 | 184.9* |

*indicates the presence of additional peaks

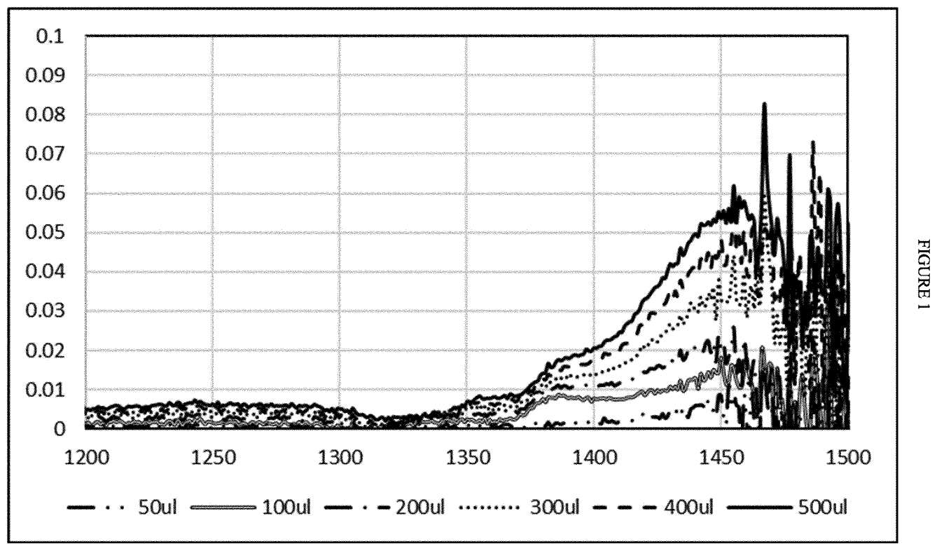

6 mL of Batch 3 was spun down to pellets and resuspended in 1 mL of methanol. Absorption spectra were measured with successively increasing amounts of this suspension added to methanol. With reference to FIG. 1, a peak at about 1450 nm is observed which increases in intensity with increasing concentration of the nanoparticles (FIG. 1 also shows absorption at about 1500 nm which is attributable to methanol).

### Photoacoustic Imaging

The PAI can be carried out using the equipment and method as described in Angewandte Chemie International Edition, 2020, the contents of which are incorporated herein by reference (https://onlinelibrary.wiley.com/doi/abs/10.1002/anie.202010228).

1. A light-absorbing agent for use in photoacoustic imaging wherein the light-absorbing agent comprises nanoparticles comprising a light-absorbing material comprising an electron accepting unit and an electron donating unit wherein the electron-donating unit is a unit of formula (IIIc-1): 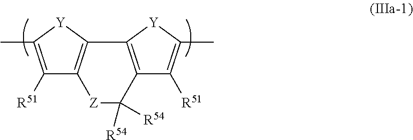
wherein:
Y in each occurrence is independently O or S;
Z in each occurrence is O, S, NR⁵⁵, or C(R⁵⁴)₂;
R⁵¹ in each occurrence is H or a substituent;
R⁵⁴ in each occurrence is independently a substituent; and
R⁵⁵ is H or a substituent.

2. The light-absorbing agent according to claim 1 wherein the light-absorbing material is a polymer; the electron-accepting unit is an electron-accepting repeat unit; and the electron donating unit is an electron-donating repeat unit.

3. The light-absorbing agent according to claim 2 wherein the light-absorbing material is a conjugated polymer.

4. The light-absorbing agent according to claim 3 wherein the conjugated polymer is a donor-acceptor polymer comprising alternating electron-donating and electron-accepting repeat units.

5. The light-absorbing agent according to claim 1 wherein the electron-accepting unit comprises a group of formula (XVI): 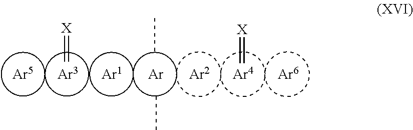
wherein Ar is a substituted or unsubstituted benzene or 6-membered heteroaromatic ring containing N and C ring atoms; Ar¹ is a substituted or unsubstituted 5- or 6-membered heteroaromatic ring containing N and C ring atoms; Ar² is a substituted or unsubstituted 5- or 6-membered heteroaromatic ring or is absent; Ar³ is a 5-membered ring or a substituted or unsubstituted 6-membered ring; Ar⁴ is a 5-membered ring or a substituted or unsubstituted 6-membered ring or is absent; Ar⁵ is a substituted or unsubstituted monocyclic or polycyclic group containing at least one aromatic or heteroaromatic ring; Ar⁶ is a substituted or unsubstituted monocyclic or polycyclic group containing at least one aromatic or heteroaromatic ring or is absent; and each X is independently a substituent bound to a carbon atom of Ar³ and, where present, Ar⁴ with the proviso that at least one X group is an electron withdrawing group.

6. The light-absorbing agent according to claim 1 wherein the nanoparticles comprise a core comprising the light-absorbing material and silica.

7. The light-absorbing agent according to claim 6 wherein the light-absorbing material is distributed within a matrix comprising the silica.

8. The light-absorbing agent according to claim 6 wherein the light-absorbing material is covalently bound to the silica.

9. The light-absorbing agent according to claim 6 wherein the light-absorbing material is not covalently bound to the silica.

10. The light-absorbing agent according to claim 1 wherein a core of the nanoparticle comprises a central region encapsulated by a shell; and the light-absorbing material is disposed in the central region.

11. The light-absorbing agent according to claim 10 wherein the shell comprises or consists of the silica.

12. The light-absorbing agent according to claim 1 wherein the light-absorbing material has a peak absorption wavelength of at least 1100 nm.

13. The light-absorbing agent according to claim 1 wherein the nanoparticles have a number average diameter of less than 200 nm as determined by dynamic light scattering.

14. A light-absorbing agent for use in photoacoustic imaging wherein the light-absorbing agent comprises nanoparticles comprising a light-absorbing material comprising an electron accepting unit and an electron donating unit wherein the electron-accepting unit is a group of formula (XVI): 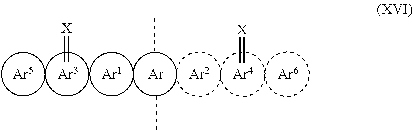
wherein Ar is a substituted or unsubstituted benzene or 6-membered heteroaromatic ring containing N and C ring atoms; Ar¹ is a substituted or unsubstituted 5- or 6-membered heteroaromatic ring containing N and C ring atoms; Ar e is a substituted or unsubstituted 5- or 6-membered heteroaromatic ring or is absent; Ar³ is a 5-membered ring or a substituted or unsubstituted 6-membered ring; Ar⁴ is a 5-membered ring or a substituted or unsubstituted 6-membered ring or is absent; Ar⁵ is a substituted or unsubstituted monocyclic or polycyclic group containing at least one aromatic or heteroaromatic ring; Ar⁶ is a substituted or unsubstituted monocyclic or polycyclic group containing at least one aromatic or heteroaromatic ring or is absent; and each X is independently a substituent bound to a carbon atom of Ar³ and, where present, Ar⁴ with the proviso that at least one X group is an electron withdrawing group.

15. A diagnostic agent for use in photoacoustic imaging comprising nanoparticles having a core comprising a light-absorbing material and silica.
